(19) United States
(12) Patent Application Publication
(10) Pub. No.: US 2024/0209097 A1
(43) Pub. Date: Jun. 27, 2024

(54) TREATMENT REGIMENS

(71) Applicant: Novartis AG, Basel (CH)

(72) Inventors: Scott ROLLINS, Oklahoma City, OK (US); Russell ROTHER, Oklahoma City, OK (US); Jonathan STOCKER, Cary, NC (US); Andreas BRUEDERLE, Basel (CH)

(21) Appl. No.: 18/516,831

(22) Filed: Nov. 21, 2023

Related U.S. Application Data
(63) Continuation of application No. 16/346,896, filed on May 2, 2019, now abandoned.
(60) Provisional application No. 62/541,133, filed on Aug. 4, 2017.

(30) Foreign Application Priority Data
Nov. 3, 2016 (GB) 1618532.4

(51) Int. Cl. C07K 16/28; A61P 7/00

(52) U.S. Cl. CPC C07K 16/2854; A61P 7/00; C07K 2317/24; C07K 2317/76

(57) ABSTRACT

The present invention relates to the treatment or prevention of P-selectin mediated disorders, and to anti-P-selectin antibodies or binding fragments thereof, for use in the treatment or prevention of such disorders. In particular, the invention relates to the treatment or prevention of pain crises associated with sickle cell disease, and to anti-P-selectin antibodies or binding fragments thereof, for use in the treatment or prevention of pain crises associated with sickle cell disease.

### SEQUENCE LISTING

The instant application contains a Sequence Listing which has been submitted electronically in XML file format and is hereby incorporated by reference in its entirety. Said XML copy, created on Apr. 20, 2023, is named PAT057533-US-CNT_ST26.SQL and is 26,326 bytes in size.

### FIELD OF INVENTION

The present invention relates to the treatment or prevention of P-selectin mediated disorders, and to anti-P-selectin antibodies or binding fragments thereof, for use in the treatment or prevention of such disorders. In particular, the invention relates to the treatment or prevention of pain crises associated with sickle cell disease, and to anti-P-selectin antibodies or binding fragments thereof, for use in the treatment or prevention of pain crises associated with sickle cell disease.

### BACKGROUND

Sickle cell disease (SCD) is one of the most common monogenic disorders worldwide caused by a homozygous or compound heterozygous mutation in the HBB gene which encodes haemoglobin. The mutation results in the production of an altered from of haemoglobin, known as HbS. Although polymerisation of deoxygenated HbS is considered to be the initiating event in the pathogenesis of SCD, it is recognised that the morbidity and mortality of the disease are primarily driven by vaso-occlusion. It has been proposed that vaso-occlusion is caused by the adhesion of sickle erythrocytes and leukocytes to the endothelium resulting in vascular obstruction and tissue ischaemia. The degree of sickle erythrocyte adhesion reportedly correlates with vaso-occlusion and increased severity of disease. Activated and adherent leukocytes likely drive vaso-occlusion in collecting venules, whereas sickle erythrocytes may facilitate the occlusion of smaller vessels or in situations where there is no potent inflammatory trigger. In addition, platelets can bind to erythrocytes, monocytes and neutrophils to form aggregates, which contribute to abnormalities of blood flow in SCD.

Sickle cell pain crisis (SCPC), also known as sickle cell-related pain crisis, or vaso-occlusive crisis, is the most common cause of healthcare encounters in SCD, resulting in a decrease in quality of life and an increase in the risk of death. The pathophysiologic origin of SCPC is thought to be a culmination of vaso-occlusion, inflammation and nociception. It has been proposed that by stopping the SCPC at an early stage, subsequent tissue and organ damage, multi-organ failure, and death may be minimised.

Although the adhesion of leukocytes during inflammation can involve multiple adhesion molecules, the process is initiated by P-selectin. P-selectin is found in storage granules of resting endothelial cells and platelets and is rapidly transferred to the cell membrane upon activation of the cell during processes such as inflammation. P-selectin expressed on the surface of the endothelium mediates abnormal rolling and static adhesion of sickle erythrocytes to the vessel surface in vitro. In addition, translocation of endothelial P-selectin to the cell surface results in prompt adhesion of sickle erythrocytes to vessels and the development of vascular occlusion in transgenic sickle cell mice. Further, activated platelets bind to neutrophils to form aggregates in a P-selectin-dependent manner.

P-selectin-mediated adhesion to the endothelium can be prevented in vitro using P-selectin-blocking agents. The adherence of sickle erythrocytes and leukocytes to the endothelium is substantially reduced when P-selectin is blocked in transgenic mice expressing human sickle haemoglobin. Doses of heparin that are sufficient to block P-selectin improve microvascular blood flow in patients with SCD. These data support the concept that endothelial and platelet P-selectin are central to multiple abnormal cell-cell adhesive interactions in SCD and suggest that blockade of P-selectin could reduce or eliminate vaso-occlusion, inflammation, and the associated SCPC.

SelG1 (Crizanlizumab) is described in WO2008/069999, which is hereby incorporated by reference in its entirety. SelG1 (Crizanlizumab) is a humanised monoclonal antibody directed against P-selectin. The antibody binds to the lectin-binding domain located in the amino terminus of P-selectin with high affinity and specificity and blocks the interaction of P-selectin with its receptor P-selectin glycoprotein ligand-1 (PSGL-1). The present invention relates to the use of P-selectin in the treatment or prevention of P-selectin mediated disorders, such as sickle cell pain crises. In particular the invention relates to a dosage regime which the inventors have found to be of a particularly good efficacy.

### BRIEF SUMMARY OF THE DISCLOSURE

In a first aspect the invention relates to an anti-P-selectin antibody or binding fragment thereof, preferably crizanlizumab or a binding fragment thereof, for use in the treatment or prevention of a P-selectin mediated disorder, wherein the antibody or binding fragment thereof is first provided in a loading phase, during which the subject receives a first amount of the antibody or binding fragment thereof over a given period of time, and then a further amount provided in a maintenance phase, during which the subject receives a lower amount of the antibody or binding fragment thereof over a given period of time.

In a second aspect the invention relates to an anti-P-selectin antibody or binding fragment thereof, preferably crizanlizumab or a binding fragment thereof, for use in the prevention of a sickle cell pain crisis, wherein the antibody or binding fragment thereof is provided to a subject at an amount of between 1 mg/kg to 20 mg/kg in one or more loading doses followed by a plurality of maintenance doses, wherein average time intervals between the maintenance doses are longer than average time intervals following the one or more loading doses, or wherein the concentration of the loading doses is higher than the concentration of the maintenance doses.

In a third aspect the invention relates to a method of treating or preventing an anti-P-selectin mediated disorder, the method comprising the step of providing a subject with an anti-P-selectin antibody or binding fragment thereof, preferably crizanlizumab or a binding fragment thereof, wherein the antibody or binding fragment thereof is first provided in a loading phase, during which the subject receives a first amount of the antibody or binding fragment thereof over a given period of time, and then further provided in a maintenance phase, during which the subject receives a lower amount of the antibody or binding fragment thereof over a given period of time.

In a fourth aspect the invention relates to a method of preventing a sickle cell pain crisis, the method comprising the step of providing a subject with an anti-P-selectin antibody or binding fragment thereof, preferably crizanlizumab or a binding fragment thereof, at an amount of between 1 mg/kg to 20 mg/kg in one or more loading doses followed by a plurality of maintenance doses, wherein average time intervals between the maintenance doses are longer than average time intervals following the one or more loading doses, or wherein the concentration of the loading doses is higher than the concentration of the maintenance doses.

In a fifth aspect the invention relates to an anti-P-selectin antibody, preferably crizanlizumab or a binding fragment thereof, for use in the prevention of a sickle cell pain crisis, wherein the antibody is first provided in a loading phase, during which the subject receives two loading doses of the antibody at an amount of 2.5 mg/kg to 5 mg/kg or 2.5 mg/kg to 7.5 mg/kg and wherein the time interval between the two loading doses is 2 weeks (+/−3 days), and then further provided in a maintenance phase, during which the subject receives a plurality of maintenance doses of the antibody at an amount of 2.5 mg/kg to 5 mg/kg or 2.5 mg/kg to 7.5 mg/kg and wherein the time interval between the plurality of maintenance doses is 4 weeks (+/−3 days). Throughout this document, time intervals defined together with “+/−3 days” also include “+/−2 days” and “+/−1 day”.

In a sixth aspect, the present invention relates to crizanlizumab or a binding fragment thereof for use in the manufacture of a medicament for prevention of a sickle cell pain crisis, wherein the antibody is first provided in a loading phase, during which the subject receives two loading doses of the antibody at an amount of between 2.5 to 7.5 mg/kg, preferably 5 mg/kg, and wherein the time interval between the two loading doses is 2 weeks (+/−3 days), and then further provided in a maintenance phase, during which the subject receives a plurality of maintenance doses of the antibody at an amount of between 2.5 mg/kg to 7.5 mg/kg, preferably 5 mg/kg, and wherein the time interval between the plurality of maintenance doses is 4 weeks (+/−3 days).

### BRIEF DESCRIPTION OF THE DRAWINGS

FIG. 1 shows Kaplan-Meier Curves for the median time to the first (FIG. 1A) and the median time to the second (FIG. 1B) sickle cell pain crisis by treatment group. The P-values are for comparison of each active treatment group with placebo with the use of the log-rank test.

FIG. 2 shows pharmacokinetic and pharmacodynamic analyses of SelG1 in patients with sickle cell disease. FIG. 2A depicts mean trough serum levels of SelG1 during the 52-week treatment phase as assessed in a capture ELISA assay. FIG. 2B shows mean trough levels of P-selectin inhibition (%) in serum during the 52-week treatment phase, as assessed in a surface plasmon resonance (SPR) assay.

FIG. 3 is a graph illustrating the effects of the anti-P-selectin antibody SelG1 on serum levels of soluble P-selectin.

### DETAILED DESCRIPTION

The present invention is based on the inventors' surprising finding that an anti-P-selectin antibody, when provided at a specific concentration and/or at specific time intervals has an exceptional ability to reduce or prevent sickle cell pain crises in patients with sickle cell disease.

Sickle cell pain crisis is associated with severe pain which may often last for as long as a few weeks. Reoccurring sickle cell pain crises may lead to tissue and/or organ damage, and in some cases result in premature death.

The inventors have shown that by providing patients with sickle cell disease with an anti-P-selectin antibody crizanlizumab, at a concentration of 1 mg/kg to 20 mg/kg in loading doses followed by 1 mg/kg to 20 mg/kg maintenance doses as described herein, particularly in an embodiment (herein and hereafter referred as “trial embodiment”), wherein crizanlizumab was administered intraveneously at a concentration of 5 mg/kg for the first two doses two weeks apart (loading dose) followed by 5 mg/kg every 4 weeks (maintenance dose), effectively preventing sickle cell pain crises by eliminating the crises event or by reducing the annual rate of sickle cell pain crises. In the trial embodiment the annual rate of sickle cell pain crises is significantly reduced by on average at least 30% up to 70% (as compared to the annual rate of sickle cell pain crises in the same patient had prior to receiving crizanlizumab treatment), Although the trial embodiment was tested in a clinical trial (SUSTAIN phase II trial NCT01895361) for 12 months, it is believed that such effect can be maintained, in particular when the patient continues receiving maintenance doses.

The inventors have found that treatment of sickle cell disease patients with the SelG1 antibody at a dose range of between 2.5 mg/kg to 5 mg/kg and at a dose interval of between 2 to 4 weeks (+/−3 days) is particularly effective.

In addition, further factors indicating the severity and status of sickle cell disease are improved in patients treated with the anti-P-selectin antibody crizanlizumab as described in the present invention. The inventors have surprisingly found that the treatment described herein, in particular the treatment described in the trial embodiment, provides the following further advantages:
increases the average time to first sickle cell pain crisis by on average between 2-4 months, to provide an average time to first sickle cell pain crisis which is three times the normal length of time;
increases the average time to second sickle cell pain crisis by on average between 1-3 months, to provide an average time to second sickle cell pain crisis which is twice the normal length of time;
reduces the number of days patients are hospitalised by on average at least 40%.

Furthermore, the inventors have unexpectedly found that the anti-P-selectin antibody crizanlizumab described herein, especially demonstrated in the trial embodiment, does not trigger an immunogenic response even during the course of a long-term treatment regimen. Antibody based therapies are usually associated with immunogenic responses, such as the development of neutralising antibodies. Such antibodies may react with the therapeutic antibody and preclude its activity as well as pose significant safety risks to patients. The development of neutralising antibodies is a particular concern for chronic diseases (such as sickle cell disease) where long-term treatment is required. However, surprisingly, in this case the inventors have found that the anti-P-selectin antibody crizanlizumab does not generate an immunogenic response in patients, making it particularly suited for long-term treatment of a chronic disease such as sickle cell disease.

Hydroxyurea has been the only commercially available treatment for sickle cell disease for the last 20 years. However, many patients on hydroxyurea continue to experience sickle cell pain crises, develop end-organ damage, and have decreased life expectancies. In addition, adherence to hydroxyurea remains a challenge and some patients are reluctant to take this medication due to safety concerns. Moreover, the treatment with hydroxyurea is only approved for the treatment of vaso-occlusion in patients with the HbSS genotype but not for other genotypes such as HbSC, HbSβ⁰−thalassemia and HbSβ⁰+thalassemia. Sickle cell patients having these other genotypes remain without adequate treatment.

Therefore, the treatment described herein provides a long needed improvement in the treatment of patients with sickle cell disease. Furthermore, as demonstrated herein, it can be used, and shows benefit, in combination with concomitant hydroxyurea treatment, if desired.

Accordingly, the present invention provides a needed treatment for all types of sickle cell disease sufferers by means of a specific dosage regime of anti-P-selectin antibody which is shown herein to surprisingly and effectively reduce sickle cell pain crises without any unwanted immunogenic responses.

### Anti-P-Selectin Antibody or Binding Fragment Thereof

The term “anti-P-selectin antibody or binding fragment thereof” as used herein refers to an antibody, or binding fragment thereof, which comprises a P-selectin binding domain. The binding of the antibody (or binding fragment thereof) to P-selectin inhibits the binding of P-selectin to PSGL-1 and thereby reduces the formation of P-selectin/PSGL-1 complexes. Suitably, the anti-P-selectin antibody or binding fragment thereof may reduce the formation of P-selectin/PSGL-1 complexes by at least 30%, at least 40%, at least 50%, at least 60%, at least 70%, at least 80%, at least 90%, at least 95%, at least 96%, at least 97%, at least 98%, at least 99%, or more as compared to a suitable control (for example a sample without the presence of an anti-P-selectin antibody or binding fragment thereof).

Additionally or alternatively, an anti-P-selectin antibody or binding thereof may dissociate preformed P-selectin/PSGL-1 complexes. In a suitable embodiment antibody or binding fragment thereof may dissociate at least 30%, at least 40%, at least 50%, at least 60%, at least 70%, at least 80%, at least 90%, at least 95%, at least 96%, at least 97%, at least 98%, at least 99%, or more of preformed P-selectin/PSGL-1 complexes. As before, this property may be compared to a suitable control (for example a sample without the presence of an anti-P-selectin antibody or binding fragment thereof).

Additionally or alternatively, an anti-P-selectin antibody or binding thereof may refer to an antibody or binding thereof that is capable of binding to P-selectin specifically, i.e. it binds to P-selectin with an affinity higher than an antibody that is well known not to bind P-selectin specifically. The affinity can be suitably determined by, for example, surface plasmon resonance (BIAcore™) assay. Ideally, the K_{d} of a P-selectin antibody or a fragment thereof is ≤1000 nM, or ≤500 nM, or ≤100 nM, or ≤50 nM, or more preferably by a K_{d}≤25 nM, and still more preferably by a K_{d}≤10 nM, and even more preferably by a K_{d}≤5 nM, or ≤1 nM, or ≤0.1 nM.

In one embodiment, the anti-P-selectin antibody or a binding fragment thereof is crizanlizumab or a binding fragment thereof.

In one embodiment, the anti-P-selectin antibody or binding fragment thereof may bind P-selectin at any suitable epitope. Suitably, the anti-P-selectin antibody or binding fragment thereof may bind an epitope which is found in the P-selectin lectin-like domain.

In one embodiment, the anti-P-selectin antibody of binding fragment thereof binds P-selectin at amino acid positions 1 to 35 of SEQ ID NO: 1. Suitably the anti-P-selectin antibody or binding fragment thereof binds P-selectin at amino acid positions 4 to 23 of SEQ ID NO: 1. More suitably, the anti-P-selectin antibody or binding fragment thereof binds P-selectin at amino acid positions 4, 14, 17, 21, and 22 of SEQ ID NO: 1.

In one embodiment, the anti-P-selectin antibody or binding fragment thereof comprises a light chain variable region having a CDR sequence selected from the group consisting of KASQSVDYDGHSYMN (SEQ ID NO: 2), AASNLES (SEQ ID NO: 3) and QQSDENPLT (SEQ ID NO: 4).

Specific features of the humanised antibody SelG1, which is a suitable antibody to be employed in the methods and medical uses of the present invention, are set out below.

In a suitable embodiment, the anti-P-selectin antibody or binding fragment thereof may comprise a light chain variable CDR with an amino acid sequence that varies from a sequence selected from the group consisting of KASQSVDYDGHSYMN (SEQ ID NO: 2), AASNLES (SEQ ID NO: 3) and QQSDENPLT (SEQ ID NO: 4) by no more than four amino acid residues, by no more than three amino acid residues, by no more than two amino acid residues, or by no more than one amino acid residue.

In one embodiment the anti-P-selectin antibody or binding fragment thereof comprises a light chain variable region comprising SEQ ID NO: 5. Suitably, the anti-P-selectin antibody or binding fragment thereof comprises of a light chain variable region consisting of SEQ ID NO: 5.

In a suitable embodiment the anti-P-selectin antibody or binding fragment thereof comprises a light chain variable region which comprises or consists of a polypeptide which is at least 90%, at least 95%, at least 96%, at least 97%, at least 98%, or at least 99% identical to SEQ ID NO: 5.

In one embodiment, the anti-P-selectin antibody or binding fragment thereof comprises a heavy chain variable region having a CDR sequence selected from the group consisting of SYDIN (SEQ ID NO: 6), WIYPGDGSIKYNEKFKG (SEQ ID NO: 7) and RGEYGNYEGAMDY (SEQ ID NO: 8).

In a suitable embodiment, the anti-P-selectin antibody or binding fragment thereof may comprise a heavy chain variable CDR with an amino acid sequence that varies from a sequence selected from the group consisting of SYDIN (SEQ ID NO: 6), WIYPGDGSIKYNEKFKG (SEQ ID NO: 7) and RGEYGNYEGAMDY (SEQ ID NO: 8) by no more than four amino acid residues, by no more than three amino acid residues, by no more than two amino acid residues, or by no more than one amino acid residue.

In one embodiment the anti-P-selectin antibody or binding fragment thereof comprises a heavy chain variable region comprising SEQ ID NO: 9. Suitably, the anti-P-selectin antibody or binding fragment thereof comprises a heavy chain variable region consisting of SEQ ID NO: 9.

In a suitable embodiment the anti-P-selectin antibody or binding fragment thereof comprises a heavy chain variable region which comprises or consists of a polypeptide which is at least 90%, at least 95%, at least 96%, at least 97%, at least 98%, or at least 99% identical to SEQ ID NO: 9.

In one embodiment the anti-P-selectin antibody or binding fragment thereof comprises a heavy chain variable region comprising three CDRs comprising, consisting essentially of or consisting of SEQ ID NO: 6, SEQ ID NO: 7, and SEQ ID NO: 8, respectively and a light chain variable region comprising three CDRs comprising, consisting essentially of or consisting of SEQ ID NO: 2, SEQ ID NO: 3, and SEQ ID NO: 4, respectively.

In one embodiment the anti-P-selectin antibody or binding fragment thereof comprises a light chain variable region comprising, consisting essentially of or consisting of the sequence SEQ ID NO: 5 and a heavy chain variable region comprising, consisting essentially of or consisting of the sequence SEQ ID NO: 9.

In a suitable embodiment, the antibody or binding fragment thereof may further comprise a constant region. The constant region may comprise a light chain constant region and/or a heavy chain constant region.

The light chain constant region may comprise a human kappa chain or a human lambda chain. Alternatively, the light chain constant region may consist of a human kappa chain or consist of a human lambda chain. Suitably the human kappa chain may be according to SEQ ID NO: 10. Alternatively the human kappa chain may be at least 90%, at least 95%, at least 96%, at least 97%, at least 98%, or at least 99% identical to SEQ ID NO: 10.

The heavy chain constant region may be selected from the group consisting of: IgG, IgA, IgD, IgE, and IgM. Immunoglobulin constant regions may be further classified into isotypes. Thus, the heavy chain constant region may be selected from the group consisting of: IgG₂, IgG₁ IgG₃ and IgG₄.

In one embodiment the heavy chain constant region may comprise an IgG. More suitably, the heavy chain constant region may comprise an IgG₂.

Alternatively the heavy chain constant region may consist of an IgG. More suitably, the heavy chain of the constant region may consist of an IgG₂. Suitably the IgG₂ may be according to SEQ ID NO: 11. Alternatively the IgG₂ may be at least 90%, at least 95%, at least 96%, at least 97%, at least 98%, or at least 99% identical to SEQ ID NO: 11. For example, an IgG₂ sequence to be employed in the invention may comprise five or less, four or less, three or less, two or less, or one or less mutations in IgG₂ sequence according to SEQ ID NO: 11. Suitably, the IgG₂ sequence to be employed in the invention may comprise one mutation in the sequence according to SEQ ID NO: 11. In such an embodiment, the IgG₂ to be employed in the invention suitably has a sequence according to SEQ ID NO: 23. An IgG₂ according to SEQ ID NO: 23 may be desirable in order to further reduce complement activation.

In one embodiment the anti-P-selectin antibody comprises a light chain which is at least 90%, at least 95%, at least 96%, at least 97%, at least 98%, at least 99% identical to SEQ ID NO: 12. Suitably, the anti-P-selectin antibody comprises a light chain according to SEQ ID NO: 12.

In one embodiment the anti-P-selectin antibody comprises a heavy chain which is at least 90%, at least 95%, at least 96%, at least 97%, at least 98%, at least 99% identical to SEQ ID NO: 13. Suitably, the anti-P-selectin antibody comprises a heavy chain according to SEQ ID NO: 13.

In a suitable embodiment the anti-P-selectin antibody comprises a light chain which is at least 90%, at least 95%, at least 96%, at least 97%, at least 98%, at least 99% identical to SEQ ID NO: 12, and a heavy chain which is at least 90%, at least 95%, at least 96%, at least 97%, at least 98%, at least 99% identical to SEQ ID NO: 13. Suitably the anti-P-selectin antibody comprises a light chain according to SEQ ID NO: 12, and a heavy chain according to SEQ ID NO: 13.

Other suitable anti-P-selectin antibodies are disclosed in WO2005/100402, WO1993/021956 and WO1994/025067, which are hereby incorporated by reference in their entirety. In one embodiment, the suitable anti-P-selectin antibody or a fragment thereof is inclacumab or a binding fragment thereof.

In the context of the present invention, the term “binding fragment” as used herein refers to a portion of an antibody capable of binding a P-selectin epitope.

In one embodiment, the binding fragment may comprise an antigen binding and/or variable region. Merely by way of example, a suitable binding fragment may be selected from the group consisting of Fab, Fab′, F(ab′)2, Fv and scFv. Suitable binding fragments may be produced by various methods known in the art. A Fab′ fragment, for example, may be produced by papain digestion of an antibody. A F(ab′)2 fragment, for example, may be produced by pepsin digestion of an antibody.

In one embodiment, the anti-P-selectin antibody or binding fragment thereof, preferably crizalizumab or a binding fragment thereof, has very low immunogenicity. More suitably, the anti-P-selectin antibody, preferably Crizanlizumab, has no or low immunogenicity. The term immunogenicity as used herein, refers to the ability of the antibody or binding fragment thereof to trigger the production of neutralising antibodies against it in the subject.

As mentioned elsewhere in this specification, the generation of neutralising antibodies is highly undesirable, as they may neutralise a therapeutic antibody (or binding fragment thereof), rendering it ineffective. The production of neutralising antibodies may result in a decrease in the levels of the therapeutic antibodies in the subject. Thus, it will be appreciated that a consistent level or amount of therapeutic antibodies in the subject (for example in a serum sample from the subject) may be indicative that no such neutralising antibodies have been produced, and thus that the therapeutic antibody has little or no immunogenicity. By the term consistent it is meant that the level of the therapeutic antibody does not fluctuate in a subject by more than 5%, more than 10%, more that 15%, more than 20%, more than 25%, more than 30%, more than 35%, more than 45%, or more than 50% during the maintenance phase.

Treatment and/or Prevention of a P-Selectin Mediated Disorder

In the context of the present invention, the term “P-selectin mediated disorder and/or symptoms” refers to a disorder and/or symptoms which is associated with increased levels of P-selectin/PSGL-1 complexes. As touched upon elsewhere in this specification, the anti-P-selectin antibody or binding fragment thereof has the ability to reduce the formation of P-selectin/PSGL-1 complexes. It may also have the ability to dissociate pre-formed P-selectin/PSGL-1 complexes. Accordingly, it will be appreciated that the use of anti-P-selectin antibodies or binding fragments thereof, allows the prevention of P-selectin mediated disorders and/or symptoms by inhibiting the formation of new P-selectin/PSGL-1 complexes. It will also be appreciated that the use of anti-P-selectin antibodies or binding fragments thereof allows the treatment of existing P-selectin mediated disorders and/or symptoms by dissociating pre-formed P-selectin/PSGL-1 complexes. Suitably, the reduction in the formation of P-selectin/PSGL-1 complexes and the dissociation of such complexes occurs during cell to cell interactions. Therefore, suitable disorders and/or symptoms prevented by the anti-P-selectin antibodies or binding fragments thereof described herein are disorders associated with increased levels of P-selectin/PSGL-1 complexes in cell to cell interactions.

Increased levels of P-selectin/PSGL-1 complexes may be observed in a wide range of disorders and/or symptoms. In particular, they are observed in subjects or samples from subjects with inflammatory and/or thrombotic disorders and/or symptoms. Thus, an anti-P-selectin antibody or binding fragment thereof may be used to treat disorders, such as inflammatory and/or thrombotic disorders selected from the group consisting of: sickle cell disease, sickle cell pain crises, arthritis (e.g., rheumatoid arthritis, osteoarthritis, and psoriatic arthritis), graft rejection, graft versus host disease, asthma, chronic obstructive pulmonary disease, psoriasis, dermatitis, sepsis, nephritis, lupus erythematosis, scleroderma, rhinitis, anaphylaxis, diabetes, multiple sclerosis, atherosclerosis, thrombosis, tumor metastasis, allergic reactions, thyroiditis, ischemic reperfusion injury (e.g., due to myocardial infarction, stroke, or organ transplantation), cancer (e.g., multiple myeloma) and conditions associated with extensive trauma, or chronic inflammation, such as, for example, type IV delayed hypersensitivity, associated for example with infection by Tubercle bacilli, or systematic inflammatory response syndrome, or multiple organ failure.

Sickle cell pain crises may be experienced by subjects with sickle cell disease. The anti-P-selectin antibody or binding fragment thereof may have particular utility in treating and preventing sickle cell pain crisis in such subjects. Suitably, the anti-P-selectin antibody or binding fragment thereof may be used to treat and/or prevent sickle cell pain crisis in subjects with sickle cell disease with a genotype selected from the group consisting of: HbSS, HbSC, HbSβ⁰−thalassemia and HbSβ⁰+thalassemia.

Successful treatment and/or prevention of a P-selectin-mediated disorder and/or symptoms, such as pain crisis associated with sickle cell disease may be demonstrated by one or more of the following parameters, each of which is able to be achieved by the methods or medical uses of the present invention. Successful treatment and/or prevention of a pain crisis associated with sickle cell disease may be demonstrated by the ability to achieve an increase in the average time to first sickle cell pain crisis by between 2-4 months, suitably by 3 months, or more.

Successful treatment and/or prevention of a pain crises associated with sickle cell disease may be demonstrated by the ability to achieve an increase in the average time to first sickle cell pain crises that is three times the length of time in an appropriate control group. Successful treatment and/or prevention of a pain crisis associated with sickle cell disease may be demonstrated by the ability to achieve an increase in the average time to a second sickle cell pain crisis by between 1-3 months, suitably by 2 months, or more. Successful treatment and/or prevention of as pain crises associated with sickle cell disease may be demonstrated by the ability to achieve an increase in the average time to a second sickle cell pain crisis that is twice the length of time in an appropriate control group. Successful treatment and/or prevention of a pain crises associated with sickle cell disease may be demonstrated by the ability to achieve a reduction in the number of days patients are hospitalised by 40% or more.

### A Subject

The term “subject” refers to a human suffering from a P-selectin mediated disorder and/or symptoms. Suitably, the subject may have sickle cell disease.

In one embodiment, the subject may be provided with an anti-P-selectin antibody or binding fragment as a first line treatment for a P-selectin mediated disorder. In such an embodiment, the subject would have not been provided any other treatment for a P-selectin mediated disorder prior to commencement of treatment in accordance with the present invention.

In another embodiment the subject may have received another treatment for a P-selectin mediated disorder and/or symptoms prior to commencement of treatment in accordance with the present invention.

In another embodiment, the subject may have had at least 2, at least 3, at least 4, at least 5, at least 6, at least 7, at least 8, at least 9, or at least 10 SCPC events within the last 12 months before start of the treatment.

### Providing

The term “providing” as used herein encompasses any techniques by which the subject receives a therapeutically effective amount of the anti-P-selectin antibody or binding fragment thereof. For example, providing may encompass administering the anti-P-selectin antibody or binding fragment thereof to a subject.

It shall be appreciated that there are various routes in which the subject may be provided with an anti-P-selectin antibody or binding fragment thereof. Such suitable routes may be selected from the group consisting of: intravenous, oral, parenteral, intraperitoneal, intramuscular, intravascular, intranasal, intraperitoneal, rectal, subcutaneous, transdermal and percutaneous. More suitably, the subject may be provided with an anti-P-selectin antibody or binding fragment thereof by intravenous route. In one embodiment, the intravenous route is by injection.

The anti-P-selectin antibody or binding fragment thereof may be provided to the subject over any reasonable delivery time. Suitable delivery times may be selected from anywhere between 1 minute to 2 hours, 5 minutes to 90 minutes, 15 minutes to 70 minutes, 20 minutes to 1 hour, or 30 minutes to 50 minutes, for example. In one embodiment, the subject may be provided with an anti-P-selectin antibody or binding fragment thereof over a delivery time of 30 minutes.

Delivery times are suitably applicable to providing the anti-P-selectin antibody or binding fragment thereof by injection, preferably injection intravenously.

In one embodiment, the anti-P-selectin antibody or binding fragment thereof may be provided to the subject by intravenous injection over 30 minutes.

In an embodiment, the anti-P-selectin antibody or binding fragment thereof may be provided to the subject in combination with a second treatment. The second treatment may be selected from any other known treatments for P-selectin mediated disorders, for example: hydroxyurea and/or erythropoietin. The combined treatment has a synergistic effect on treating the P-selectin mediated disorder.

In one embodiment, the anti-P-selectin antibody or binding fragment thereof is provided in combination with hydroxyurea.

The anti-P-selectin antibody or binding fragment thereof may be provided in any form to a subject. Suitably it may be provided in the form of a pharmaceutical composition. Suitable pharmaceutically acceptable carriers, diluents and excipients for formulating a pharmaceutical composition of an anti-P-selectin antibody or binding fragment are defined elsewhere in this specification.

### Loading Phases and Maintenance Phases

Certain aspects of the present invention refer to the provision of an antibody or binding fragment, preferably crizanlizumab or a binding fragment thereof, to a subject in a loading phase, followed by further provision of the antibody or binding fragment, preferably crizanlizumab or a binding fragment thereof, in a maintenance phase. In such an embodiment, the subject receives a first amount of the antibody or binding fragment over a given period of time during the loading phase, and then receives a lower amount of the antibody or binding fragment over a given period of time, suitably the same given period of time, during the maintenance phase. The different amounts of the antibody required by the loading phase and the maintenance phase may be provided by providing different doses of the antibody and/or by employing different intervals of time between administrations of the antibody. For example, during the maintenance phase the antibody may be provided at essentially the same dose as used during the loading phase, but with longer intervals of time between each incidence of administration. Alternatively, the time between intervals of administration may be the same in each of the loading and maintenance phases, but the dose of antibody provided in each incidence of administration during the maintenance phase may be lower.

Merely by way of example, a suitable loading phase may involve the provision to the subject of a suitable antibody, or binding fragment thereof, in an amount of approximately 2.5 mg/kg per week of the loading phase (whether this is provided weekly, bi-weekly, or otherwise). In such an embodiment of the invention a suitable maintenance phase may involve the provision to the subject of the antibody, or binding fragment thereof, in an amount of approximately 1.25 mg/kg per week of the maintenance phase (for example by provision bi-weekly or every four weeks). Generally the amount of the antibody, or binding fragment thereof, provided per week of a loading phase may be approximately double that provided per week of the maintenance phase.

As another example, a suitable loading phase may involve the provision to the subject of a suitable antibody, or binding fragment thereof, in an amount of approximately 3.75 mg/kg per week of the loading phase (whether this is provided weekly, bi-weekly, or otherwise). In such an embodiment of the invention a suitable maintenance phase may involve the provision to the subject of the antibody, or binding fragment thereof, in an amount of approximately 1.875 mg/kg per week of the maintenance phase (for example by provision bi-weekly or every four weeks). Generally the amount of the antibody, or binding fragment thereof, provided per week of a loading phase may be approximately double that provided per week of the maintenance phase.

It will be appreciated that, since the length of the maintenance phase may be much longer than the loading phase, the total amount of the antibody or binding fragment received by the subject over the maintenance phase may be much more than that provided during the relatively shorter loading phase. However, the amount of the antibody that the subject will receive over a set period of the maintenance phase will be lower than the amount that would be received over the same period of the loading phase.

The loading and maintenance phases required by such embodiments of the invention may be put into practice by use of the loading and maintenance doses, and associated administration regimens, considered below.

Various aspects of the invention refer to the average time intervals between maintenance doses, and to average time intervals following the one or more loading doses. It will be recognised that suitable average time intervals may be achieved by varying the number of doses, and the individual intervals between maintenance doses, or following loading doses (whether the loading dose in question is followed by a further loading dose, or a maintenance dose). The following paragraphs provide examples of suitable individual time intervals that may be used in achieving a desired average time interval.

### Loading Dose

In the context of the present invention, the term loading dose refers to an amount of anti-P-selectin antibody or binding fragment thereof, preferably crizanlizumab or a binding fragment thereof, provided to the subject during the initial stages of the treatment. The purpose of a loading dose is to more rapidly achieve therapeutic levels of the antibody or binding fragment thereof in the subject than would be reached with maintenance dosing only. In addition, the loading dose can also achieve sufficient levels of antibody to enable therapeutic levels to be maintained once the switch to maintenance dosing is made. Furthermore, the loading dose allows a relatively constant therapeutic level of the antibody or binding fragment to be achieved in which the antibody or binding fragment is in a steady-state. The steady state of the antibody or binding fragment may be regarded as a state in which the overall intake of the antibody or binding fragment thereof is in dynamic equilibrium with its elimination. Once the therapeutic levels of the antibody or binding fragment thereof are reached, the loading doses may be followed by a plurality of maintenance doses.

That a therapeutic level of the antibody or binding fragment thereof has been established may be determined by directly assessing the concentration of the therapeutic agent in the subject's circulation.

Therapeutic levels of the antibody or binding fragment thereof may be more rapidly achieved and maintained through providing the subject with one or more loading doses. These may be calculated with reference to the weight of the subject. Suitably, the subject may be provided with one, two, three, four, or more loading doses. More suitably the subject may be provided with two loading doses. The concentration of the loading doses may be between 1 mg/kg to 20 mg/kg, or between 1 mg/kg to 10 mg/kg, or between 2.5 mg/kg to 7.5 mg/kg or between 2.5 mg/kg to 5 mg/kg. More suitably, the concentration of the loading doses may be 2.5 mg/kg, 3 mg/kg, 3.5 mg/kg, 4 mg/kg, 4.5 mg/kg, 5 mg/kg, or 7.5 mg/kg. More suitably, the concentration of the loading doses may be 2.5 mg/kg, 5 mg/kg, 7.5 mg/kg or 10 mg/kg. More suitably the concentration of the loading doses is 7.5 mg/kg. Most suitably the concentration of the loading doses is 5 mg/kg.

The skilled person will appreciate that the number of loading doses may depend on the concentration of the loading doses. Thus if the concentration of the loading doses is lower, a greater number of loading doses may be required in order to achieve therapeutic levels of the antibody or binding fragment thereof.

By the same token, if the concentration of the loading doses is increased, fewer loading doses may be required in order to achieve therapeutic levels of the antibody or binding fragment thereof. In some embodiments, only one loading dose may be required. In one embodiment the time intervals between the loading doses may be between 1 and 4 weeks (+/−3 days), more suitably between 1 and 3 weeks (+/−3 days). More suitably, the time intervals between the loading doses may be 2 weeks (+/−3 days). More suitably, the time intervals between the loading doses may be 1 week (+/−1 days). Suitably the loading doses are distributed at regular intervals during the loading phase.

Increasing the loading dose concentration may allow the time intervals between the loading doses to be extended. By way of example, the time intervals between the loading doses may be 2 weeks (+/−3 days), 3 weeks (+/−3 days), 4 weeks (+/−3 days), 5 weeks (+/−3 days), 6 weeks (+/−3 days). Suitably, the time interval between the loading doses may be 4 weeks (+/−3 days) or 2 weeks (+/−3 days).

### Maintenance Dose

In the context of the present invention, the term maintenance dose refers to an amount of anti-P-selectin antibody or binding fragment thereof, preferably crizanlizumab or a binding fragment thereof, provided to the subject in order to maintain therapeutic levels of the antibody or binding fragment thereof. Such therapeutic levels of the antibody or binding fragment thereof are achieved and maintained more rapidly by providing the patient with one or more loading doses as described above prior to providing the maintenance doses.

Suitably, the maintenance doses maintain a relatively constant therapeutic level of the antibody or binding fragment in a subject throughout the maintenance phase. Suitably, the maintenance doses of the antibody or binding fragment maintain the antibody or binding fragment in a steady state in the subject throughout the maintenance phase.

The period during which maintenance doses are provided will depend on the length of time in which it is desired to maintain therapeutic levels of the antibody or binding fragment thereof. This in turn will depend on the P-selectin mediated disorder to be treated or prevented. By way of example, maintenance doses may be provided over a period of at least 3 months, at least 6 months, at least 9 months, at least 12 months, at least 16 months, at least 20 months, at least 24 months, or more. In some instances (for example when disorder to be treated is a life-long condition) the maintenance doses may be provided throughout the subject's life.

In a suitable embodiment, when the disorder to be prevented is a sickle cell pain crisis, the maintenance doses may be provided for at least 3 months, at least 6 months, at least 12 months, or more. More suitably, the maintenance doses may be provided for at least 12 months. As sickle cell disease is a life-long condition, the maintenance doses may be provided throughout the subject's life.

The total number of maintenance doses provided will depend on the duration of treatment. This in turn will depend on the P-selectin mediated disorder to be treated or prevented. The total number of maintenance doses may be at least 3, at least 6, at least 12 or more. Suitably the total number of maintenance doses is between 3 and 24, between 6 and 18, or between 9 and 15. In one embodiment, the total number of maintenance doses is 12.

Suitably the maintenance doses are distributed at regular intervals over the duration of treatment.

Suitably, the time interval between the maintenance doses may be 6 weeks (+/−3 days), 5 weeks (+/−3 days), 4 weeks (+/−3 days), 3 weeks (+/−3 days), 2 weeks (+/−3 days), or 1 week (+/−3 days). Suitably the time interval between maintenance doses is 4 weeks (+/−3 days),

In a suitable embodiment the time intervals between the maintenance doses may be longer than time intervals between the loading doses.

More suitably the time interval between the loading doses may be 4 weeks (+/−3 days). Suitably, when the time interval between maintenance doses is 4 weeks (+/−3 days), the time interval between loading doses may be 2 weeks (+/−3 days).

In one embodiment, the time intervals between the loading doses may be the same as the time intervals between the maintenance doses.

Suitable maintenance doses may be determined with reference to the subject's body weight. In a suitable embodiment the concentration of the maintenance doses may be between 1 mg/kg to 10 mg/kg, or between 2.5 mg/kg to 5 mg/kg, or between 2.5 mg/kg to 7.5 mg/kg. More suitably, the concentration of the maintenance doses may be 2.5 mg/kg, 3 mg/kg, 3.5 mg/kg, 4 mg/kg, 4.5 mg/kg, 5 mg/kg, or 7.5 mg/kg. More suitably, the concentration of the maintenance doses may be 2.5 mg/kg, 5 mg/kg, or 7.5 mg/kg. More suitably the concentration of the maintenance doses is 7.5 mg/kg. Most suitably the concentration of the maintenance doses is 5 mg/kg.

In one embodiment the maintenance doses may be provided every 4 weeks at a concentration of 2.5 mg/kg, 5 mg/kg, or 7.5 mg/kg.

In a suitable embodiment, the concentration of a maintenance dose is between 1 mg/kg to 10 mg/kg or between 2.5 mg/kg to 5 mg/kg or between 2.5 mg/kg to 7.5 mg/kg, and the time interval between the maintenance doses is 4 weeks (+/−3 days). More suitably, the concentration of a maintenance dose is 2.5 mg/kg or 5 mg/kg, and the time interval between the maintenance doses is 4 weeks (3+/−days). However, it will be appreciated that the time interval between the maintenance doses may depend on the concentration of the antibody or binding fragment thereof contained in the doses. Thus if the concentration of a maintenance dose is increased, the time interval between doses may be increased. By the same token, if the concentration of a maintenance dose is decreases, the time interval between doses may be decreased.

In one embodiment the concentration of the loading doses may be higher than the concentration of the maintenance doses.

The time interval between the last loading dose and the first maintenance dose is always equal to the time interval between the maintenance doses. In the preferred embodiment, such interval is 4 weeks (+/−3 days).

### Determining Effectiveness

In another aspect, the inventors have surprisingly found that providing anti-P-selectin antibodies or binding fragments, preferably crizanlizumab or a binding fragment thereof, to subjects with P-selectin mediated disorders may lower the levels of soluble P-selectin in a sample from the subject. The inventors believe that this finding may be of utility in determining and/or monitoring the effectiveness of an anti-P-selectin antibody or binding fragment thereof treatment in a subject with a P-selectin mediated disorder and/or symptoms, for example sickle cell pain crisis.

Suitable P-selectin mediated disorders in which the method of the above aspect may be applied are any P-selectin mediated disorders in which soluble P-selectin is present. Suitably, the method may be applied in any P-selectin mediated disorders in which soluble P-selectin is elevated for example in conditions in which platelets and/or endothelial cells are activated. Such disorders may be selected from the group consisting of sickle cell disease, hyperlipidaemia, hypertension, ischemic heart disease, atherosclerosis, peripheral arterial occlusive disease, postangioplasty restenosis and ischemia/reperfusion injury.

Accordingly, the invention relates to a method of determining effectiveness of treatment with an anti-P-selectin antibody or binding fragment thereof, preferably crizanlizumab or a binding fragment thereof, the method comprising the steps of:
measuring levels of soluble P-selectin in a sample from a subject provided with an anti-P-selectin antibody or binding fragment thereof, preferably crizanlizumab or a binding fragment thereof, and
comparing the subject's soluble P-selectin levels to a reference value, and
thereby determining the effectiveness of the treatment.

In one embodiment, the method is for determining effectiveness of treatment with an anti-P-selectin antibody or binding fragment thereof in a subject with sickle cell disease.

It will be appreciated that the reference value may be based upon the levels of soluble P-selectin measured either in one or more samples from a control individual(s), or in one or more samples from a subject(s) diagnosed with a P-selectin mediated disorder and who did not receive treatment.

In one embodiment, the reference value is based upon the soluble P-selectin levels measured in one or more samples from control individual(s), which is a healthy person with normal level of soluble P-selectin. In such an embodiment, if the levels of soluble P-selectin in a sample from a subject treated for a P-selectin mediated disorder approximates the soluble P-selectin levels measured in a sample(s) from the control individual(s), it provides an indication that the treatment is effective.

By the same token, if the soluble P-selectin levels measured in a sample from a subject treated for a P-selectin mediated disorder does not approximate the soluble P-selectin levels measured in a sample from a control individual, it may be an indication that the treatment is not effective. In the context of the present disclosure the term “approximates” may be taken as referring to soluble P-selectin levels which are within at least 30%, at least 25%, at least 20%, at least 15%, at least 10%, at least 5% or less from the reference value.

In another embodiment, the reference value is based upon the levels of soluble P-selectin measured in a sample(s) from an individual(s) with a P-selectin mediated disorder who has not received treatment. Suitably, the individual with a P-selectin mediated disorder may be the subject themselves prior to receiving treatment. In such an embodiment, if the soluble P-selectin levels measured in a sample from a subject treated for a P-selectin mediated disorder approximates the soluble P-selectin levels measured in a sample from an individual with a P-selectin mediated disorder prior to receiving treatment, it may be an indication that the treatment is not effective.

By the same token, if the levels of soluble P-selectin measured in the sample from the subject treated for a P-selectin mediated disorder are lower than the levels measured in the sample from an individual with a P-selectin mediated disorder prior to receiving treatment, it may be an indication that the treatment is effective. It will be appreciated that any reduction in soluble P-selectin levels as compared to a reference value based upon soluble P-selectin levels in a sample from an individual with a P-selectin mediated disorder prior to receiving treatment may be an indication that the treatment is effective. Suitably, soluble P-selectin levels that are at least 5%, at least 10%, at least 15%, at least 20%, at least 25%, at least 30%, or more, lower than the reference value, may be an indication that the treatment is effective.

In the context of the present disclosure, the term sample refers to any suitable sample in which soluble P-selectin levels may be detected. Suitably, the sample is a fluid sample selected from the group consisting of: a blood sample (for example, a whole blood sample, a blood plasma sample, or a serum sample) and a urine sample. More suitably, the sample is a serum sample.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The reader's attention is directed to all papers and documents which are filed concurrently with or previous to this specification in connection with this application and which are open to public inspection with this specification, and the contents of all such papers and documents are incorporated herein by reference.

In one aspect the present invention provides an anti-P-selectin antibody or binding fragment thereof, for use in the treatment or prevention of a P-selectin mediated disorder in a subject, wherein the first two doses of said antibody or binding fragment thereof is provided 2 weeks (+/−3 days) apart followed by further doses provided every 4 weeks (+/−3 days), wherein each dose is between 2.5 mg per kg body weight (2.5 mg/kg) to 20 mg/kg, preferably 2.5 mg/kg to 10 mg/kg, preferably 2.5 mg/kg to 7.5 mg/kg and preferably wherein the interval between the last loading dose and the first maintenance dose is 4 weeks (+/−3 days). In one preferred embodiment, the loading dose is 5 mg/kg, the maintenance dose is 5 mg/kg, and the time interval between the last loading and first maintenance dose is 4 weeks (+/−3 days).

In another aspect, the present invention provides a method of reducing the frequency of SCPC comprising administrating an therapeutically effective amount of an anti-P-selectin antibody or binding fragment thereof to a subject in need thereof, wherein the first two doses of said antibody or binding fragment thereof is provided 2 weeks (+/−3 days) apart followed by further doses provided every 4 weeks (+/−3 days), wherein each dose is between 2.5 mg/kg to 20 mg/kg, or between 2.5 mg/kg to 10 mg/kg, or between 2.5 mg/kg to 7.5 mg/kg.

In one embodiment, each of said doses is 2.5 mg/kg. In another embodiment, each of said doses is 5 mg/kg. In another embodiment, each of said doses is 7.5 mg/kg. When the dose is initially 7.5 mg/kg, the dose is allowed to be reduced for safety reasons to 5 mg/kg at any time after the loading dose, normally 1 month, 2 months, 3 months, 4 months, 5 months, 6 months, 7 months, 8 months, 9 months, 10 months, or 11 months after the last loading dose. Safety parameters are monitored by health care professionals during the treatment. In doing so, the criteria can include, but not necessarily limit to, those indicated in Table 7.

### EMBODIMENTS OF THE PRESENT INVENTION

### Embodiments (a)

1a. An anti-P-selectin antibody or binding fragment thereof, for use in the treatment or prevention of a P-selectin mediated disorder, wherein the antibody or binding fragment thereof is first provided in a loading phase, during which the subject receives a first amount of the antibody or binding fragment thereof over a given period of time, and then a further amount of the antibody or binding fragment thereof provided in a maintenance phase, during which the subject receives a lower amount of the antibody or binding fragment thereof over a given period of time.

2a. An anti-P-selectin antibody or binding fragment thereof, for use according to embodiment 1a, wherein the antibody or binding fragment thereof binds P-selectin at an epitope corresponding to amino acids 1 to 35 of SEQ ID NO: 1.

3a. An anti-P-selectin antibody or binding fragment thereof, for use according to embodiments 1a or 2a, wherein the antibody or binding fragment thereof comprises a heavy chain sequence which is at least 90% identical to SEQ ID NO: 13.

4a. An anti-P-selectin antibody or binding fragment thereof, for use according to embodiments 1a to 3a, wherein the antibody or binding fragment thereof comprises a light chain sequence which is at least 90% identical to SEQ ID NO: 12.

5a. An anti-P-selectin antibody or binding fragment thereof, for use according to embodiments 1a to 4a, wherein the antibody comprises a heavy chain sequence which is at least 90% identical to SEQ ID NO: 13 and a light chain sequence which is at least 90% identical to SEQ ID NO: 12.

6a. An anti-P-selectin antibody or binding fragment thereof, for use according to embodiments 1a to 5a, wherein the P-selectin-mediated disorder is sickle cell pain crisis.

7a. An anti-P-selectin antibody or binding fragment thereof, for use according to embodiments 1a to 6a, wherein the anti-P-selectin antibody or binding fragment thereof is provided to the subject by an intravenous route.

8a. An anti-P-selectin antibody or binding fragment thereof, for use according to embodiments 1a to 7a, wherein the antibody or binding fragment thereof is provided to the subject at an amount of between 2.5 mg/kg to 5 mg/kg.

9a. An anti-P-selectin antibody or binding fragment thereof, for use according to embodiment 8a, wherein the antibody or binding fragment thereof is provided to the subject at an amount of 5 mg/kg.

10a. An anti-P-selectin antibody or binding fragment thereof, for use according to embodiments 1a to 9a, wherein the antibody or binding fragment thereof is provided to the subject in two loading doses.

11a. An anti-P-selectin antibody or binding fragment thereof, for use according to embodiments 1a to 10a, wherein the time interval between the loading doses is 2 weeks (+/−3 days).

12a. An anti-P-selectin antibody or binding fragment thereof, for use according to embodiments 1a to 11a, wherein the time interval between the maintenance doses is 4 weeks (+/−3 days).

13a. An anti-P-selectin antibody or binding fragment thereof, for use according to embodiments 1a to 12a further in combination with hydroxyurea.

14a. An anti-P-selectin antibody or binding fragment thereof, for use according to embodiments 1a to 13a, wherein the antibody or binding fragment thereof has low immunogenicity.

15a. An anti-P-selectin antibody or binding fragment thereof, for use according to embodiments 1a to 14a, wherein said antibody is Crizanlizumab.

### Embodiments (b)

1b. An anti-P-selectin antibody or binding fragment thereof, for use in the prevention of a sickle cell pain crisis, wherein the antibody or binding fragment thereof is provided to a subject at an amount of between 1 mg/kg to 20 mg/kg in one or more loading doses followed by a plurality of maintenance doses, wherein average time intervals between the maintenance doses are longer than average time intervals following the one or more loading doses, or wherein the concentration of the loading doses is higher than the concentration of the maintenance doses.

2b. An anti-P-selectin antibody or binding fragment thereof, for use according to embodiment 1b, wherein the antibody or binding fragment thereof binds P-selectin at an epitope corresponding to amino acids 1 to 35 of SEQ ID NO: 1.

3b. An anti-P-selectin antibody or binding fragment thereof, for use according to embodiments 1b or 2b, wherein the antibody or binding fragment thereof comprises a heavy chain sequence which is at least 90% identical to SEQ ID NO: 13.

4b. An anti-P-selectin antibody or binding fragment thereof, for use according to embodiments 1b to 3b, wherein the antibody or binding fragment thereof comprises a light chain sequence which is at least 90% identical to SEQ ID NO: 12.

5b. An anti-P-selectin antibody or binding fragment thereof, for use according to embodiments 1b to 4b, wherein the antibody comprises a heavy chain sequence which is at least 90% identical to SEQ ID NO: 13 and a light chain sequence which is at least 90% identical to SEQ ID NO: 12.

6b. An anti-P-selectin antibody or binding fragment thereof, for use according to embodiments 1b to 5b, wherein the anti-P-selectin antibody or binding fragment thereof is provided to the subject by an intravenous route.

7b. An anti-P-selectin antibody or binding fragment thereof, for use according to embodiments 1b to 6b, wherein the antibody or binding fragment thereof is provided to the subject at an amount of between 2.5 mg/kg to 5 mg/kg.

8b. An anti-P-selectin antibody or binding fragment thereof, for use according to embodiment 7b, wherein the antibody or binding fragment thereof is provided to the subject at an amount of 5 mg/kg.

9b. An anti-P-selectin antibody or binding fragment thereof, for use according to embodiments 1b to 8b, wherein the antibody or binding fragment thereof is provided to the subject in two loading doses.

10b. An anti-P-selectin antibody or binding fragment thereof, for use according to embodiments 1b to 9b, wherein the time interval between the loading doses is 2 weeks (+/−3 days).

11b. An anti-P-selectin antibody or binding fragment thereof, for use according to embodiments 1b to 10b, wherein the time interval between the maintenance doses is 4 weeks (+/−3 days).

12b. An anti-P-selectin antibody or binding fragment thereof, for use according to embodiments 1b to 11b, wherein the prevention of a sickle cell pain crisis is in a subject having sickle cell disease selected from the genotypes: HbSS, HbSC, HbSβ⁰−thalassemia and HbSβ⁰+thalassemia.

13b. An anti-P-selectin antibody or binding fragment thereof, for use according to embodiments 1b to 12b further in combination with hydroxyurea.

14b. An anti-P-selectin antibody or binding fragment thereof, for use according to embodiments 1b to 13b, wherein the antibody or binding fragment thereof has low immunogenicity.

15b. An anti-P-selectin antibody or binding fragment thereof, for use according to embodiments 1b to 14b, wherein said antibody is Crizanlizumab.

### Embodiments (c)

1c. A method of treating or preventing an P-selectin mediated disorder, the method comprising the step of providing a subject with an anti-P-selectin antibody or binding fragment thereof, wherein the antibody or binding fragment thereof is first provided in a loading phase, during which the subject receives a first amount of the antibody or binding fragment thereof over a given period of time, and then further provided in a maintenance phase, during which the subject receives a lower amount of the antibody or binding fragment thereof over a given period of time.

2c. A method according to embodiment 1c, wherein the antibody or binding fragment thereof binds P-selectin at an epitope corresponding to amino acids 1 to 35 of SEQ ID NO: 1.

3c. A method according to embodiments 1c or 2c, wherein the antibody or binding fragment thereof comprises a heavy chain sequence which is at least 90% identical to SEQ ID NO: 13.

4c. A method according to embodiments 1c to 3c, wherein the antibody or binding fragment thereof comprises a light chain sequence which is at least 90% identical to SEQ ID NO: 12.

5c. A method according to embodiments 1c to 4c, wherein the antibody comprises a heavy chain sequence which is at least 90% identical to SEQ ID NO: 13 and a light chain sequence which is at least 90% identical to SEQ ID NO: 12.

6c. A method according to embodiments 1c to 5c, wherein the P-selectin-mediated disorder is sickle cell pain crisis.

7c. A method according to embodiments 1c to 6c, wherein the anti-P-selectin antibody or binding fragment thereof is provided to the subject by an intravenous route.

8c. A method according to embodiments 1c to 7c, wherein the antibody or binding fragment thereof is provided to the subject at an amount of between 2.5 mg/kg to 5 mg/kg.

9c. A method according to embodiment 8c, wherein the antibody or binding fragment thereof is provided to the subject at an amount of 5 mg/kg.

10c. A method according to embodiments 1c to 9c, wherein the antibody or binding fragment thereof is provided to the subject in two loading doses.

11c. A method according to embodiments 1c to 10c, wherein the time interval between the loading doses is 2 weeks (+/−3 days).

12c. A method according to embodiments 1c to 11c, wherein the time interval between the maintenance doses is 4 weeks (+/−3 days).

13c. A method according to embodiments 1c to 12c further in combination with hydroxyurea. 14c. A method according to embodiments 1c to 13c, wherein the prevention of a sickle cell pain crisis is in a subject having sickle cell disease selected from the genotypes: HbSS, HbSC, HbSβ⁰−thalassemia and HbSβ⁰+thalassemia.

15c. A method according to embodiments 1c to 14c, wherein the antibody or binding fragment thereof has low immunogenicity.

16c. A method according to embodiments 1c to 15c, wherein said antibody is Crizanlizumab.

### Embodiments (d)

1d. A method of preventing a sickle cell pain crisis, the method comprising the step of providing a subject with an anti-P-selectin antibody or binding fragment thereof at an amount of between 1 mg/kg to 20 mg/kg in one or more loading doses followed by a plurality of maintenance doses, wherein average time intervals between the maintenance doses are longer than average time intervals following the one or more loading doses, or wherein the concentration of the loading doses is higher than the concentration of the maintenance doses.

2d. An anti-P-selectin antibody or binding fragment thereof, for use according to embodiment 1d, wherein the antibody or binding fragment thereof binds P-selectin at an epitope corresponding to amino acids 1 to 35 of SEQ ID NO: 1.

3d. An anti-P-selectin antibody or binding fragment thereof, for use according to embodiments 1d or 2d, wherein the antibody or binding fragment thereof comprises a heavy chain sequence which is at least 90% identical to SEQ ID NO: 13.

4d. An anti-P-selectin antibody or binding fragment thereof, for use according to embodiments 1d to 3d, wherein the antibody or binding fragment thereof comprises a light chain sequence which is at least 90% identical to SEQ ID NO: 12.

5d. An anti-P-selectin antibody or binding fragment thereof, for use according to embodiments 1d to 4d, wherein the antibody comprises a heavy chain sequence which is at least 90% identical to SEQ ID NO: 13 and a light chain sequence which is at least 90% identical to SEQ ID NO: 12.

6d. An anti-P-selectin antibody or binding fragment thereof, for use according to embodiments 1d to 5d, wherein the anti-P-selectin antibody or binding fragment thereof is provided to the subject by an intravenous route.

7d. An anti-P-selectin antibody or binding fragment thereof, for use according to embodiments 1d to 6d, wherein the antibody or binding fragment thereof is provided to the subject at an amount of between 2.5 mg/kg to 5 mg/kg.

8d. An anti-P-selectin antibody or binding fragment thereof, for use according to embodiment 7d, wherein the antibody or binding fragment thereof is provided to the subject at an amount of 5 mg/kg.

9d. An anti-P-selectin antibody or binding fragment thereof, for use according to embodiments 1d to 8d, wherein the antibody or binding fragment thereof is provided to the subject in two loading doses.

10d. An anti-P-selectin antibody or binding fragment thereof, for use according to embodiments 1d to 9d, wherein the time interval between the loading doses is 2 weeks (+/−3 days).

11d. An anti-P-selectin antibody or binding fragment thereof, for use according to embodiments 1d to 10d, wherein the time interval between the maintenance doses is 4 weeks (+/−3 days).

12d. An anti-P-selectin antibody or binding fragment thereof, for use according to embodiments 1d to 11d further in combination with hydroxyurea.

13d. An anti-P-selectin antibody or binding fragment thereof, for use according to embodiments 1d to 12d, wherein the prevention of a sickle cell pain crisis is in a subject having sickle cell disease selected from the genotypes: HbSS, HbSC, HbSβ⁰−thalassemia and HbSβ⁰+thalassemia.

14d. An anti-P-selectin antibody or binding fragment thereof, for use according to embodiments 1d to 13d, wherein the antibody or binding fragment thereof has low immunogenicity.

15d. A method according to embodiments 1d to 14d, wherein said antibody is Crizanlizumab.

### Embodiments (e)

1e. An anti-P-selectin antibody, for use in the prevention of a sickle cell pain crisis, wherein the antibody is first provided in a loading phase, during which the subject receives two loading doses of the antibody at an amount of between 2.5 mg/kg to 5 mg/kg and wherein the time interval between the two loading doses is 2 weeks (+/−3 days), and then further provided in a maintenance phase, during which the subject receives a plurality of maintenance doses of the antibody at an amount of between 2.5 mg/kg to 5 mg/kg and wherein the time interval between the plurality of maintenance doses is 4 weeks (+/−3 days).

2e. An anti-P-selectin antibody for use according to embodiment 1e, wherein the antibody binds P-selectin at an epitope corresponding to amino acids 1 to 35 of SEQ ID NO: 1.

3e. An anti-P-selectin antibody for use according to embodiments 1e or 2e, wherein the antibody comprises a heavy chain sequence which is at least 90% identical to SEQ ID NO: 13.

4e. An anti-P-selectin antibody for use according to embodiments 1e to 3e, wherein the antibody comprises a light chain sequence which is at least 90% identical to SEQ ID NO: 12.

5e. An anti-P-selectin for use according to embodiments 1e to 4e, wherein the antibody comprises a heavy chain sequence which is at least 90% identical to SEQ ID NO: 13 and a light chain sequence which is at least 90% identical to SEQ ID NO: 12.

6e. An anti-P-selectin for use according to embodiments 1e to 5e, wherein the antibody consists of a heavy chain sequence which is at least 90% identical to SEQ ID NO: 13 and a light chain sequence which is at least 90% identical to SEQ ID NO: 12.

7e. An anti-P-selectin antibody for use according to embodiments 1e to 6e, wherein the anti-P-selectin antibody is provided to the subject by an intravenous route.

8e. An anti-P-selectin antibody for use according to embodiments 1e to 7e, further in combination with hydroxyurea.

9e. A anti-P-selectin antibody for use according to embodiments 1e to 8e, wherein the prevention of a sickle cell pain crisis is in a subject having sickle cell disease selected from the genotypes: HbSS, HbSC, HbSβ⁰−thalassemia and HbSβ⁰+thalassemia.

10e. An anti-P-selectin antibody for use according to embodiments 1e to 9e, wherein the antibody or binding fragment thereof has low immunogenicity.

11e. An anti-P-selectin antibody for use according to embodiments 1e to 10e, wherein said antibody is Crizanlizumab.

### Embodiments (f)

1f. An anti-P-selectin antibody or binding fragment thereof, for use in the treatment or prevention of a P-selectin mediated disorder in a subject, wherein the first two doses of said antibody or binding fragment thereof is provided 2 weeks (+/−3 days) apart followed by further doses provided every 4 weeks (+/−3 days), wherein each dose is between 2.5 mg per kg body weight (2.5 mg/kg) to 20 mg/kg.

2f. A method of reducing the frequency of SCPC comprising administrating a therapeutically effective amount of an anti-P-selectin antibody or binding fragment thereof to a subject in need thereof, wherein the first two doses of said antibody or binding fragment thereof is provided 2 weeks (+/−3 days) apart followed by further doses provided every 4 weeks (+/−3 days), wherein each dose is between 2.5 mg/kg to 20 mg/kg.

3f. An anti-P-selectin antibody or binding fragment thereof, for use according to embodiments 1f or 2f, wherein each of said doses is 2.5 mg/kg.

4f. An anti-P-selectin antibody or binding fragment thereof, for use according to embodiments 1f or 2f, wherein each of said doses is 5 mg/kg.

5f. An anti-P-selectin antibody or binding fragment thereof, for use according to embodiments 1f or 2f, wherein each of said doses is 7.5 mg/kg.

6f. An anti-P-selectin antibody or binding fragment thereof, for use according to embodiment 5f, wherein the dose is reduced to 5 mg/kg in the course of treatment for safety reasons.

7f. An anti-P-selectin antibody or binding fragment thereof, for use according to any one of the embodiments 1f to 6f, wherein the anti-P-selectin antibody or binding fragment thereof is provided to the subject intravenously.

8f. An anti-P-selectin antibody or binding fragment thereof, for use according to any one of the embodiments 1f to 7f, wherein the P-selectin-mediated disorder is sickle cell disease.

9f. An anti-P-selectin antibody or binding fragment thereof, for use according to any one of the embodiments 1f to 8f, wherein the P-selectin-mediated disorder is sickle cell pain crisis.

10f. An anti-P-selectin antibody or binding fragment thereof, for use according to embodiment 8f or 9f, wherein the subject has HbSS genotype.

11f. An anti-P-selectin antibody or binding fragment thereof, for use according to any one of the embodiments 1f to 10f, wherein said subject has received, is receiving, or will receive hydroxyurea.

12f. An anti-P-selectin antibody or binding fragment thereof, for use according to any one of the embodiments 1f to 11f, wherein the antibody or binding fragment thereof binds P-selectin at an epitope corresponding to amino acids 1 to 35 of SEQ ID NO: 1.

13f. An anti-P-selectin antibody or binding fragment thereof, for use according to any one of the embodiments 1f to 12f, wherein the antibody is humanised.

14f. An anti-P-selectin antibody or binding fragment thereof, for use according to any one of the embodiments 1f to 13f, wherein anti-P-selectin antibody or binding fragment thereof comprises a heavy chain variable region comprising three CDRs consisting essentially of or consisting of SEQ ID NO: 6, SEQ ID NO: 7, and SEQ ID NO: 8, respectively and a light chain variable region comprising three CDRs consisting essentially of or consisting of SEQ ID NO: 2, SEQ ID NO: 3, and SEQ ID NO: 4, respectively.

15f. An anti-P-selectin antibody or binding fragment thereof, for use according to any one of the embodiments 1f to 14f, wherein the anti-P-selectin antibody or binding fragment thereof comprises a light chain variable region comprising, consisting essentially of or consisting of the sequence SEQ ID NO: 5 and a heavy chain variable region comprising, consisting essentially of or consisting of the sequence SEQ ID NO: 9.

16f. An anti-P-selectin antibody or binding fragment thereof, for use according to any one of the embodiments 1f to 15f, wherein said antibody is Crizanlizumab.

17f. An anti-P-selectin antibody or binding fragment thereof, for use in freeing the SCD patients of SCPC event for at least one year.

18f. An anti-P-selectin antibody or binding fragment thereof, for use in elongating time to first SCPC on-treatment by at least one month.

19f. An anti-P-selectin antibody or binding fragment thereof, for use according to embodiments 17f to 18f, wherein said antibody is Crizanlizumab.

### Examples

### 1 Methods

### 1.1 Patients

Male or female patients with a confirmed medical diagnosis of sickle cell disease (SCD) (HbSS, HbSC, HbSβ⁰−thalassemia, HbSβ⁺-thalassemia or other genotypes) between the ages of 16 and 65 years of age who had experienced between 2 and 10 sickle cell pain crises (SCPC) in the 12 months preceding enrolment in the study were eligible. Patients on hydroxyurea had been on the drug for at least 6 months, with a stable dose for at least 3 months, and the dose should not have been altered during the 52-week treatment phase, except for safety reasons. In patients not on hydroxyurea at the Screening Visit, hydroxyurea therapy should not have been initiated throughout the 52-week treatment phase. Patients could not be receiving chronic red blood cell transfusion therapy. A full listing of the inclusion and exclusion criteria are provided in Table 1.

### 1.2 Study Design

The study consisted of a 30-day screening phase, a 52-week treatment phase, and a 6-week follow-up evaluation phase. Randomisation was performed centrally in a 1:1:1 ratio with stratification according to number of historical SCPC in the preceding year (2-4 or 5-10) and concomitant hydroxyurea use (yes or no) during the treatment phase of the study; patients were assigned, by means of an interactive web/voice-response system, to receive either placebo, 2.5 mg/kg SelG1 (low-dose) or 5.0 mg/kg SelG1 (high-dose). Patients received infusions of placebo or SelG1 on Day 1 and Day 15±3 days (loading doses) and then every 4 weeks (maintenance dose) through week 50 for a total of 14 doses.

The last visit of the treatment phase occurred at Week 52. During the treatment phase, efficacy, immunogenicity, safety and pharmacokinetic/pharmacodynamic assessments were completed for each patient before the initial dose (Day 1), 2 weeks following the initial dose (Day 15, Week 2), every four weeks through Week 50, and at Week 52. Beginning with the Day 1 dose, patients were scheduled to make a total of 15 visits over a period of 52 weeks while receiving study drug. For the follow-up evaluation phase, patients returned for assessment at Week 58 (eight weeks after the last treatment dose).

### 1.3 Determination of Clinical Efficacy

The primary clinical efficacy end point was the annual rate of SCPC. An SCPC was defined as an acute episode of pain, with no other medically determined cause than a vaso-occlusive event, which required a medical facility visit and treatment with oral or parenteral narcotics, or a parenteral non-steroidal anti-inflammatory drug. Acute chest syndrome, hepatic sequestration, splenic sequestration and priapism (requiring a visit to a medical facility) were also considered to be SCPC. To ensure consistency across all sites, all crisis events identified by study investigators were adjudicated by an independent, blinded Crisis Review Committee (CRC) comprised of 3 independent haematologists who specialise in the treatment of SCD (a complete list of the members of this committee is provided in the Supplementary Appendix). The primary efficacy analysis was performed on adjudicated data with randomisation factors of hydroxyurea therapy and categorised crisis (SCPC) history as strata.

Secondary efficacy assessments included annual rate of days hospitalised (defined as the number of days hospitalised multiplied by 365 divided by the end date minus the date of randomisation plus one), time to first and second SCPC, and annual rate of uncomplicated SCPC. All efficacy analyses were performed on the data collected during the 52-week treatment phase. Trough SelG1 serum concentrations (pharmacokinetics), measured at baseline and weeks 6, 10, 14, 18, 22, 26, 30, 34, 38, 42, 46, 50, 52 and 58, and percent inhibition of P-selectin/PSGL-1 binding (pharmacodynamics), measured at the same time points, were also assessed.

### 1.4 Safety Assessment

Safety assessments were performed during the screening phase, at specified times prior to and following administration of study drug during the 52-week treatment phase, and during the follow-up evaluation phase through the final week 58 visit. Safety assessments included: physical examination; vital signs (blood pressure, pulse rate, respiratory rate, oxygen saturation and oral body temperature); immunogenicity, clinical laboratory tests (chemistry panel, complete blood count with reticulocyte count, urinalysis, prothrombin time/international normalized ratio, activated partial thromboplastin time, pregnancy tests and haptoglobin); 12-lead electrocardiogram; and reported or observed AEs. Adverse events were coded with the use of preferred terms from the Medical Dictionary for Regulatory Activities (MedDRA) (www.msso.org/MSSOWeb/index.htm) and tabulated as incidence rates in the three treatment arms.

### 1.5 Statistical Analysis

A sample size of at least 50 patients per arm provided the study with a statistical power of greater than 90%, at an alpha level of 0.05, to detect a reduction of 40% (i.e., a change from 3.0 to 1.8) in annual rate of SCPC, assuming the mean annual rate of SCPC is 3.0 with a standard deviation of 1.7 in the placebo arm.

For the primary end point, the analysis was performed according to the intention-to-treat principle with the use of data on all 198 patients who underwent randomisation; reduction in the annual rate of SCPC in the high-dose arm was analysed with the use of the Wilcoxon rank-sum test. A hierarchical testing procedure was employed (a=0.05 for high-dose vs. placebo, and if significant, low-dose vs. placebo) and randomisation was stratified by historical SCPC in the prior year (2-4 or 5-10) and concomitant hydroxyurea use (yes or no). To assess the effect of treatment on the annual rates of uncomplicated SCPC, and days hospitalised, the Wilcoxon rank-sum test was used. The log-rank test was used to compare times to the first and second SCPC in the three treatment arms.

### 1.6 Determining Immunogenicity

Immunogenicity was assessed using an AlphaLISA platform, where SelG1 is directly coupled to both donor and acceptor beads. Patient serum is mixed with donor and acceptor beads. The presence of antibodies in the serum directed against SelG1 would bridge the donor and acceptor beads, resulting in a measurable fluorescence output.

### 1.7 Measuring Soluble P-Selectin

Levels of soluble P-selectin (sP-selectin) were assessed using a human sP-selectin Immunoassay in a 1.25-hour solid phase ELISA. This assay employs the quantitative sandwich immunoassay technique. A monoclonal antibody specific for sP-Selectin has been pre-coated onto a microplate. Standards, patient serum samples and control are pipetted into the wells together with a polyclonal antibody specific for sP-Selectin which has been conjugated to horseradish peroxidase. After removal of unbound conjugated antibody, a substrate is added and color is developed which is proportional to analyte concentration.

### 2 Results

### 2.1 Patients

One hundred and ninety eight patients (89 men and 109 women) at 60 sites in the United States, Brazil, and Jamaica met the eligibility criteria and were randomly assigned to receive high-dose SelG1 (67 patients), low-dose SelG1 (66 patients), or placebo (65 patients) between August 2013 and January 2015. Of the patients who underwent randomisation, 129 completed the study. Patients that discontinued early from the study were balanced between the three-treatment arms. All 198 randomised patients were included in the intention-to-treat analyses. The baseline characteristics and laboratory values of the patients in the three treatment arms were similar (Table 2).

### 2.2 Pharmacokinetics, Pharmacodynamics and Immunogenicity of SelG1

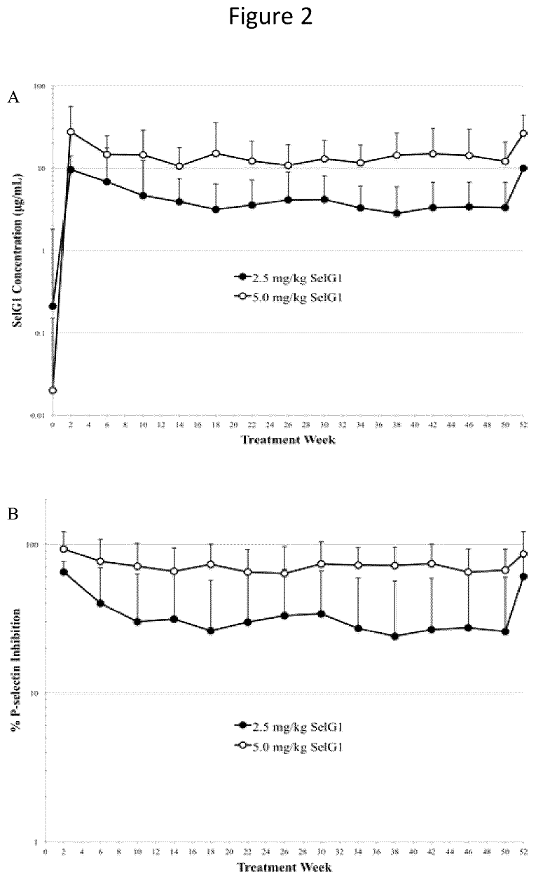

High-dose SelG1 administered once every 4 weeks (+3 days), after 2 biweekly loading doses, effectively blocked P-selectin/PSGL-1 binding, throughout the treatment phase of the study, while low-dose SelG1 demonstrated partial blockade (FIG. 2). Antibodies against SelG1 were not detected in any treated patient.

### 2.3 Clinical Efficacy

### 2.3.1 Primary End Point

At the end of the treatment phase, the median annual rates of SCPC were 1.63 in the high-dose arm versus 2.98 in the placebo arm (45.3% reduction, P=0.01) (Table 3). The drug effect appeared to be dose-dependent as the median rate of SCPC in the low-dose arm was 2.01 (32.6% reduction versus the placebo arm, P=0.18). The number of patients with an SCPC rate of zero at the end of the study was 24 of 67 in the high-dose arm (35.8%), 12 of 66 in the low-dose arm (18.2%), and 11 of 65 in the placebo arm (16.9%). Table 4 indicates the post-hoc analysis of SCPC event-free patients subpopulations. Treatment with SelG1 5.0 mg/kg appears to increase the likelihood of adult patients with SCD being SCPC event-free while on treatment, even in high-risk subpopulations. SelG1 5.0 mg/kg was also effective in those who had experienced at least two SCPCs in the previous year despite taking HU, suggesting that this dose is effective as a disease-modifying agent that meets an unmet medical need.

The robustness of the primary end point outcome was evaluated in an analysis of the annual rate of SCPC in the per protocol population. The median annual rates of SCPC were 1.04 in the high-dose arm versus 2.18 in the placebo arm (52.3% reduction, P=0.018). The number of patients who had no SCPC throughout the 52-week treatment phase in the per protocol population was 15 of 40 in the high-dose arm (37.5%) and 5 of 41 in the placebo arm (12.2%).

The annual rates of SCPC were evaluated based on receipt of concomitant hydroxyurea and SCD genotype (HbSS or other genotypes). The median annual rates of SCPC in patients on concomitant hydroxyurea were 2.43 in the high-dose arm versus 3.58 in the placebo arm (32.1% reduction, P=0.084), while the rates of SCPC in patients not on concomitant hydroxyurea were 1.00 in the high-dose arm versus 2.00 in the placebo arm (50.0% reduction, P=0.046). The median annual rates of SCPC in patients with HbSS genotype were 1.97 in the high-dose arm versus 3.01 in the placebo arm (34.6% reduction, P=0.060) and the rates of SCPC in patients with other genotypes (HbSC, HbSβ⁰−thalassemia, HbSβ⁺-thalassemia and other genotypes) were 0.99 in the high-dose arm versus 2.00 in the placebo arm (50.5% reduction, P=0.22). The magnitude of the differences between the high-dose arm and the placebo arm in the different subgroups was similar to that observed for the primary analysis, but generally did not reach statistical significance as these subgroup analyses were not powered to do so.

### 2.3.2 Secondary End Points

The median annual rates of days hospitalised for the high-dose arm and the placebo arm were 4.00 and 6.87, respectively; a 41.8% reduction with SelG1 treatment, although the treatment difference was non-significant (P=0.450) due to the variability of the data (Table 5).

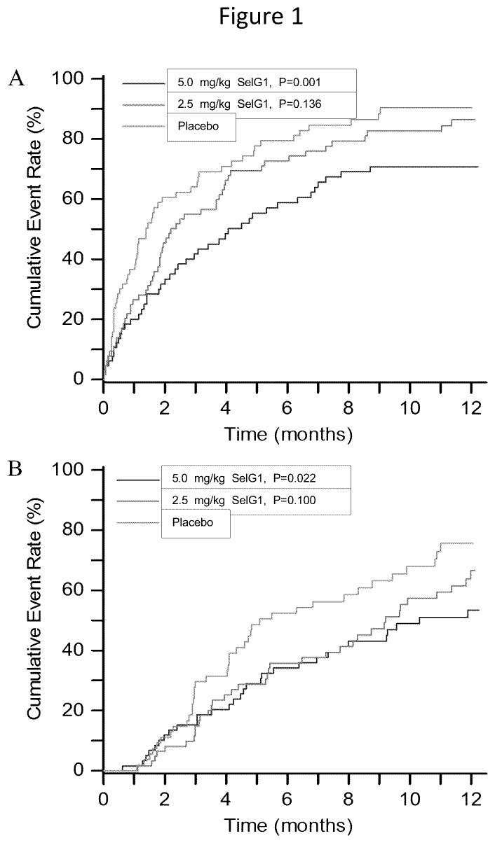

The median time to the first SCPC was significantly longer in patients treated with high-dose SelG1 relative to those treated with placebo (4.07 vs. 1.38 months, P=0.001), as was the median time to the second SCPC (10.32 vs. 5.09 months, P=0.022) (Table 5). The decrease in the frequency of SCPC with 5.0 mg/kg SelG1 treatment was evident within 2 weeks of treatment and the effect was maintained throughout the 52-week treatment phase (FIG. 1A).

In almost all the subpopulations evaluated, SelG1 5.0 mg/kg increased the time to first SCPC versus placebo by two-fold or greater (Table 6). The effect was present in both SCPC subgroups (2-4 and 5-10 SCPCs in the previous year). The largest treatment difference was observed in patients with the HbSS genotype, with a 3.7-fold increase in time to first SCPC observed for SelG1 5.0 mg/kg versus placebo (4.1 vs 1.1 months; Hazard ratio: 0.50). It is also notable that in patients taking HU who experienced 2-10 SCPCs in the previous year, the time to first on-study SCPC was longer with SelG1 5.0 mg/kg versus placebo (2.4 vs 1.2 months; Hazard ratio: 0.58).

Altogether, treatment with SelG1 5.0 mg/kg significantly delayed the time to first SCPC on-treatment in adults living with SCD compared with placebo in most of the subpopulations investigated, including patients with the HbSS genotype, suggesting a potential for disease modification. SelG1 5.0 mg/kg was also effective in those individuals taking HU who had still experienced 2-10 SCPCs in the previous year, indicating a synergistic treatment effect of P-selectin inhibition.

The median times to first and second SCPC were also longer in patients treated with 2.5 mg/kg SelG1 relative to placebo, but these differences were not significant (FIGS. 1A and 1B, Table 5). The annual rate of uncomplicated SCPC in the high-dose arm vs. the placebo arm was reduced by 62.9% (medians of 1.08 vs. 2.91, P=0.015).

### 2.3.3 Soluble P-Selectin Levels

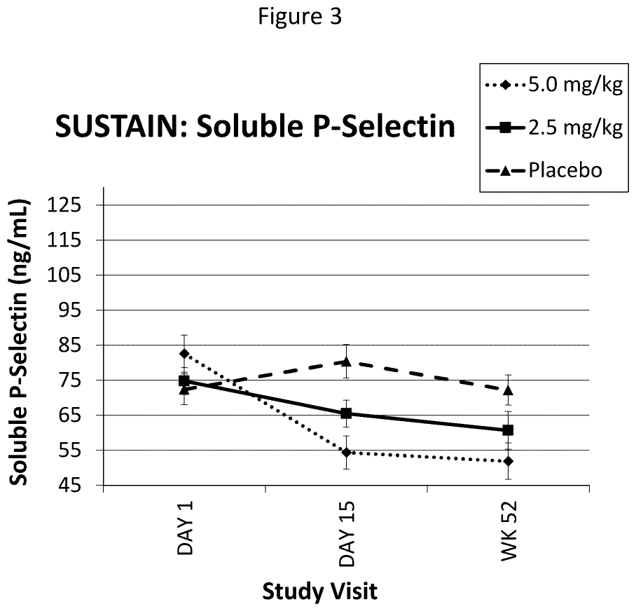

A soluble form of P-selectin is derived either from alternative mRNA splicing that generates an isoform that lacks the transmembrane domain and/or from proteolytic cleavage of the membrane bound form of P-selectin. Soluble P-selectin has been proposed as useful biomarker for various pathologic states involving activation of platelets and/or endothelial cells. Treatment with SelG1 was found to significantly decrease soluble P-selectin levels in patients with sickle cell disease. The decrease in soluble P-selectin is consistent in the clinical improvements noted in the treated patients (FIG. 3).

### 2.4 Safety

Serious adverse events were reported in 55 patients: 17 in the high-dose arm, 21 in the low-dose arm and 17 in the placebo arm (Table 7). Serious adverse events occurring in at least 2 patients on active treatment were pyrexia, endocarditis, influenza, pneumonia and urinary tract infection. Adverse events that occurred in 10% or more of patients in either active dose arm were arthralgia, pruritus, vomiting, chest pain, diarrhoea, road traffic accident, fatigue, myalgia, musculoskeletal chest pain, abdominal pain, influenza and oropharyngeal pain. There were no apparent increases in infections with SelG1 treatment. Five patients died during the study, 2 in the high-dose arm, 1 in the low-dose arm and 2 in the placebo arm; no deaths were deemed related to study drug. Three additional single-occurrence adverse events that were considered both serious and life-threatening but did not result in death include sepsis (placebo arm), anemia (low-dose arm), and intracranial hemorrhage (low-dose arm).

### 3 Conclusion

Despite an increased understanding of the pathophysiology of SCD, the treatment options for disease-related complications remain limited. Hydroxyurea, approved by the US Food and Drug Administration (FDA) in 1998, remains the only drug that has been shown in peer-reviewed studies to modify the natural history of SCD (Charache S et al, NEJM, 1995). However, many patients on hydroxyurea continue to experience acute painful episodes, develop end-organ damage, and have decreased life expectancies (Steinberg M H et al, JAMA, 2003). In addition, adherence to hydroxyurea treatment remains a challenge (Candrilli S D et al, Am J hematol, 2011) and some patients are reluctant to take this medication due to safety concerns. Recent drug trials have failed to show statistically significant and clinically meaningful results (Heeney M M et al, N Engl J Med, 2016; Machado, R F Blood, 2011; Ataga, K I Br. J. Haematol, 2011.), attesting to the complexity of disease pathophysiology and the difficulty in developing successful therapeutic interventions in SCD.

In this phase 2 multinational, placebo-controlled study, we observed that treatment with high-dose SelG1 resulted in a 46% reduction in the annual rate of SCPC relative to placebo treatment. In addition, the median times to first and second SCPC were two to three times longer in patients treated with high-dose when compared to placebo. These treatment effects were statistically significant with effect sizes indicating clinical significance. Smaller, non-significant differences in the annual rates of SCPC and times to first and second SCPC were observed in patients treated with low-dose SelG1 compared with those treated with placebo, indicating a dose-dependent effect and providing further confirmation that P-selectin blockade reduces vaso-occlusion in SCD.

The clinical efficacy of SelG1 in this study is especially noteworthy because it provides a therapeutic option for patients with SCD who experience frequent acute pain episodes despite the use of hydroxyurea as well as those patients who either fail hydroxyurea therapy or refuse to take it. While this study was not powered to detect statistical differences within subgroups of the population enrolled, the magnitude of the reduction in the annual rate of SCPC in patients treated with high-dose SelG1 versus those treated with placebo appeared to be clinically meaningful regardless of hydroxyurea use during the study. Therefore, SelG1 may not only provide therapeutic benefit as a single agent, but may provide additive, or perhaps synergistic effects in combination with hydroxyurea in decreasing the frequency of pain episodes in patients with SCD.

Hydroxyurea is approved for the treatment of the complications of vaso-occlusion in HbSS patients and has not been adequately tested in other genotypes. Patients with all common SCD genotypes were enrolled and treated in the SUSTAIN study, with genotypes other than HbSS observed in 29% of the ITT population. The magnitude in reduction of the annual rate of SCPC with SelG1 treatment versus placebo treatment was 34.6% in HbSS patients and 50.5% in genotypes other than HbSS, indicating that SELG1 is beneficial to patients with HbSS and other SCD genotypes.

SelG1 was well tolerated during the study, with no increase in the incidence of serious adverse events in the active treatment groups compared with placebo. The adverse events that occurred in 10% or more of patients in the active dose arms and were elevated over the placebo arm by at least 2-fold were arthralgia, pruritus, vomiting, chest pain and diarrhoea. There were no apparent increases in infections. Although five deaths occurred during the study, none were deemed related to the study drug. In addition to a low prevalence of adverse effects, no patients developed a detectable immune response against SelG1 during the study. This characteristic of SelG1 is critical for the ability to administer it as a chronic therapy in this disease setting.

In conclusion, the P-selectin inhibitor, SelG1, significantly reduced the frequency of sickle cell pain crises in patients with SCD in a dose-dependent fashion and appeared to be safe and well tolerated. Clinically meaningful reductions in SCPC with SelG1 were observed regardless of concomitant hydroxyurea usage or SCD genotype. Chronic inhibition of P-selectin with once a month IV dosing of SelG1 represents a potentially new disease-modifying treatment option for patients with SCD, one that broadens the very limited therapeutic armamentarium against this orphan disease.

### 4 Further Clinical Study

### 4.1 Objective

This Phase II open-label, single-arm, multicenter study is conducted in 10-15 sites in the USA. To identify at least 27 patients with evaluable PK/PD data, up to 45 patients are enrolled and receive crizanlizumab 5.0 mg/kg; subsequently, 10 additional patients are enrolled to receive crizanlizumab 7.5 mg/kg as an exploratory objective. Patients aged 18-70 years with SCD of any genotype, and who have experienced at least one vaso-occlusion (VOC) in the year prior to study entry, are eligible for inclusion. Concomitant use of hydroxyurea is only permitted if the patient has been using it for ≥6 months and plans to continue at a stable dose and schedule for the duration of the study. Crizanlizumab is administered in the clinic by intravenous infusion every 4 weeks, with an additional loading dose after the first 2 weeks, for at least 2 years or until discontinuation. Dose interruptions and dose reductions (from 7.5 mg/kg to 5.0 mg/kg only) are allowed if necessary for safety reasons. All patients undergo complete PK/PD and immunogenicity sampling when they receive the first dose (week 1, day 1: pre-dose to 24 hours post-dose, and on days 4, 8 and 15) and fifth dose (week 15, day 1: pre-dose to 24 hours post-dose, and on days 4, 8, 15, 22 and 29 thereafter). Pre-dose PK, PD and immunogenicity assessments are performed at each additional study visit until week 51; from week 51, immunogenicity assessments is performed every 24 weeks. The final safety follow-up visit takes place 105 days after the last dose of study treatment. An interim analysis is performed when there are at least 27 patients in the crizanlizumab 5.0 mg/kg group with single-dose evaluable PK profiles, at least five of whom have single- and multiple-dose evaluable PK profiles.

### 4.3 Results

The primary objective is to characterize the PK/PD profile of crizanlizumab 5.0 mg/kg using: area-under-the-curve [AUC] to day 15, steady-state AUC calculated to the end of a dosing interval, and maximum observed drug concentration (Cₘₐₓ) as PK parameters; and AUC to day 15 and at steady state as PD parameters. Other PK (e.g. time to Cₘₐₓ, drug elimination rate, and half-life) and PD (e.g. P-selectin inhibition at each time point and P-selectin inhibition time profiles) parameters are analyzed as secondary variables. The secondary objectives are to assess the efficacy, safety and tolerability of crizanlizumab.

TABLE 2
Characteristics and Baseline Values
of the Patients - ITT Population.

| | SelG1 | | SelG1 | | | |
|---|---|---|---|---|---|---|
| | 5.0 mg/kg | | 2.5 mg/kg | | Placebo | |
| Characteristic | (N = 67) | | (N = 66) | | (N = 65) | |
| Sex - no. (%) | | | | | | |
| Male | 32 | (47.8) | 30 | (45.5) | 27 | (41.5) |
| Female | 35 | (52.2) | 36 | (54.5) | 38 | (58.5) |
| Race* - no. (%) | | | | | | |
| American Indian or | 0 | | 0 | | 0 | |
| Alaskan Native | | | | | | |
| Asian | 0 | | 0 | | 0 | |
| Black, or | 60 | (89.6) | 62 | (93.9) | 60 | (92.3) |
| African American | | | | | | |
| Native Hawaiian or | 0 | | 0 | | 0 | |
| Other Pacific Islander | | | | | | |
| White | 4 | (6.0) | 2 | (3.0) | 3 | (4.6) |
| Other | 3 | (4.5) | 2 | (3.0) | 2 | (3.1) |
| Age - year | | | | | | |
| Median | 29 | | 29 | | 26 | |
| Range | 16-63 | | 17-57 | | 16-56 | |
| Sickle cell disease | | | | | | |
| genotype - no. (%) | | | | | | |
| HbSS | 47 | (70.1) | 47 | (71.2) | 47 | (72.3) |
| HbSC | 9 | (13.4) | 15 | (22.7) | 8 | (12.3) |
| HbSβ⁰ - thalassemia | 3 | (4.5) | 2 | (3.0) | 7 | (10.8) |
| HbSβ⁺ - thalassemia | 7 | (10.4) | 2 | (3.0) | 1 | (1.5) |
| Other† | 1 | (1.5) | 0 | | 2 | (3.1) |
| Concomitant | | | | | | |
| hydroxyurea | | | | | | |
| use - no. (%) | | | | | | |
| Yes | 42 | (62.7) | 41 | (62.1) | 40 | (61.5) |
| No | 25 | (37.3) | 25 | (37.9) | 25 | (38.5) |
| Categorized | | | | | | |
| number of SCPC | | | | | | |
| during previous | | | | | | |
| 12 months - no. (%) | | | | | | |
| 2-4 | 42 | (62.7) | 41 | (62.1) | 41 | (63.1) |
| 5-10 | 25 | (37.3) | 25 | (37.9) | 24 | (36.9) |
| Baseline values - | | | | | | |
| mean (SD) | | | | | | |
| Haemoglobin (g/L) | 90.8 | (17.6) | 91.8 | (18.6) | 90.3 | (15.3) |
| Haemoglobin F (%) | 12.0 | (8.9) | 10.3 | (8.4) | 11.5 | (9.1) |
| Lactate | 396.2 | (171.9) | 393.4 | (188.1) | 410.3 | (216.8) |
| dehydrogenase (U/L) | | | | | | |
| Reticulocytes (×10⁹/L) | 209.2 | (108.4) | 191.8 | (91.2) | 201.1 | (89.8) |
| Leukocytes (×10⁹/L)‡ | 9.0 | (3.8) | 8.8 | (2.8) | 8.9 | (3.7) |
| Platelets (×10⁹/L)‡ | 349.1 | (156.5) | 342.6 | (129.1) | 325.5 | (125.9) |

TABLE 3
Annual Rates of Sickle Cell Pain Crises

| | SelG1 | SelG1 | |
|---|---|---|---|
| | 5.0 mg/kg | 2.5 mg/kg | Placebo |
| Primary end point*, | | | |
| ITT population | | | |
| n | 67 | 66 | 65 |
| P value† | 0.010 | 0.180 | |
| Median rate of SCPC | 1.63 (0.00 | 2.01 (1.00 | 2.98 (1.25 |
| per year (IQR‡) | to 3.97) | to 3.98) | to 5.87) |
| Reduction vs. placebo | 45.3% | 32.6% | |
| Number of patients with | 24 | 12 | 11 |
| an SCPC rate of zero at | | | |
| the end of the study | | | |
| Primary analysis, | | | |
| PP population | | | |
| n | 40 | 44 | 41 |
| P value† | 0.018 | 0.130 | |
| Median rate of SCPC | 1.04 (0.00 | 2.00 (1.00 | 2.18 (1.96 |
| per year (IQR‡) | to 3.42) | to 3.02) | to 4.96) |
| Reduction vs. placebo | 52.3% | 8.3% | |
| Number of patients with | 15 | 7 | 5 |
| an SCPC rate of zero at | | | |
| the end of the study | | | |
| Subgroup analyses, | | | |
| ITT population | | | |
| Concomitant hydroxyurea | 42 | 41 | 40 |
| use (yes), n | | | |
| Median rate of SCPC | 2.43 (0.00 | 2.00 (1.00 | 3.58 (1.13 |
| per year (IQR‡) | to 4.01) | to 3.93) | to 6.23) |
| Reduction vs. placebo | 32.1% | 44.1% | |
| Concomitant hydroxyurea | 25 | 25 | 25 |
| use (no), n | | | |
| Median rate of SCPC | 1.00 (0.00 | 2.16 (1.89 | 2.00 (1.63 |
| per year (IQR‡) | to 2.00) | to 3.98) | to 3.90) |
| Reduction vs. placebo | 50.0% | 0% | |
| Categorized number of SCPC | 42 | 41 | 41 |
| during previous 12 months | | | |
| (2 to 4), n | | | |
| Median rate of SCPC | 1.14 (0.00 | 2.00 (1.00 | 2.00 (1.00 |
| per year (IQR‡) | to 3.96) | to 3.02) | to 3.90) |
| Reduction vs. placebo | 43.0% | 0% | |
| Categorized number of SCPC | 25 | 25 | 24 |
| during previous 12 months | | | |
| (5 to 10), n | | | |
| Median rate of SCPC | 1.97 (0.00 | 3.02 (2.00 | 5.32 (2.01 |
| per year (IQR‡) | to 3.98) | to 5.19) | to 11.05) |
| Reduction vs. placebo | 63.0% | 43.2% | |
| Sickle cell disease | 47 | 47 | 47 |
| genotype (HbSS), n | | | |
| Median rate of SCPC | 1.97 (0.00 | 2.05 (1.00 | 3.01 (1.01 |
| per year (IQR‡) | to 3.96) | to 4.96) | to 6.00) |
| Reduction vs. placebo | 34.6% | 31.9% | |
| Sickle cell disease | 20 | 19 | 18 |
| genotype (excluding | | | |
| HbSS)§, n | | | |
| Median rate of SCPC | 0.99 (0.00 | 2.00 (1.00 | 2.00 (1.86 |
| per year (IQR‡) | to 4.01) | to 3.03) | to 5.00) |
| Reduction vs. placebo | 50.5% | 0.0% | |

TABLE 4
SCPC event-free patients by prior
SCPC events, genotype and HU use

| | | |
|---|---|---|
| SelG1 | SelG1 | |
| 5.0 mg/kg | 2.5 mg/kg | Placebo |
| N = 67 | N = 66 | N = 65 |

SCPC events in the year prior to study

| | | | |
|---|---|---|---|
| 2-4 | n = 17/42 (40.5) | n = 10/41 (24.4) | n = 10/41 (24.4) |
| 5-10 | n = 7/25 (28.0) | n = 2/25 (8.0) | n = 1/24 (4.2) |

Genotype

| | | | |
|---|---|---|---|
| HbSS | n = 15/47 (31.9) | n = 9/47 (19.1) | n = 8/47 (17.0) |
| Non-HbSS | n = 9/20 (45.0) | n = 3/19 (15.8) | n = 3/18 (16.7) |

HU use

| | | | |
|---|---|---|---|
| Yes | n = 14/42 (33.3) | n = 9/41 (22.0) | n = 7/40 (17.5) |
| No | n = 10/25 (40.0) | n = 3/25 (12.0) | n = 4/25 (16.0) |

n = number of patients in subpopulation who experienced no SCPC during the study/total number of patients in subpopulation. The ratio is presented as percent in the parentheses.

TABLE 5
Secondary End Points (ITT Population)

| | SelG1 | SelG1 | |
|---|---|---|---|
| | 5.0 mg/kg | 2.5 mg/kg | Placebo |
| | (N = 67) | (N = 66) | (N = 65) |
| Annual rate of days | | | |
| hospitalized | | | |
| P value* | 0.450 | 0.837 | |
| Median rate of days | 4.00 (0.00 | 6.87 (0.00 | 6.87 (0.00 |
| hospitalized per | to 25.72) | to 18.00) | to 28.30) |
| year (IQR†) | | | |
| Reduction vs. placebo | 41.8% | 0.0% | |
| Time to first SCPC | | | |
| P value‡ | 0.001 | 0.136 | |
| Median time to first | 4.07 (1.31 | 2.20 (0.95 | 1.38 (0.39 |
| SCPC in months (IQR†) | to NR)§ | to 6.60) | to 4.90) |
| Hazard ratio (95% CI) | 0.495 (0.331, | 0.752 (0.515, | |
| | 0.741) | 1.097) | |
| Time to second SCPC | | | |
| P value‡ | 0.022 | 0.100 | |
| Median time to second | 10.32 (4.47 | 9.20 (3.94 | 5.09 (2.96 |
| SCPC in months (IQR†) | to NR)§ | to 12.16) | to 11.01) |
| Hazard ratio (95% CI) | 0.534 (0.329, | 0.693 (0.440, | |
| | 0.866) | 1.092) | |
| Annual rate of | | | |
| uncomplicated SCPC | | | |
| P value* | 0.015 | 0.120 | |
| Median rate of | 1.08 (0.00 | 2.00 (0.00 | 2.91 (1.00 |
| uncomplicated SCPC | to 3.96) | to 3.025) | to 5.00) |
| per year (IQR†) | | | |
| Reduction vs. placebo | 62.9% | 31.3% | |

*For the comparison between active treatment groups and placebo during treatment, calculated with the use of a stratified Wilcoxon rank-sum test;
†Interquartile range;
‡For the comparison between the high-dose (i.e. 5.0 mg/kg) SelG1 group and placebo during treatment, calculated with the use of the log-rank test;
§Not reportable. The 75% value for the interquartile range was not achieved within the 52-week study.

TABLE 6
Median time to first SCPC (in months) by
prior SCPC events, genotype and HU use

| | | |
|---|---|---|
| SelG1 | | |
| 5.0 mg/kg | Placebo | HR |
| N = 67 | N = 65 | (95% CI)* |

SCPC events in the year prior to study

| | | | |
|---|---|---|---|
| 2-4 | n = 25/42 | n = 31/41 | 0.53 |
| | 4.8^{†} (1.8-NR) | 1.6 (0.6-6.7) | (0.31, 0.90) |
| 5-10 | n = 18/25 | n = 23/24 | 0.47 |
| | 2.4^{†} (1.3-7.8) | 1.0 (0.3-3.0) | (0.25, 0.89) |

Genotype

| | | | |
|---|---|---|---|
| HbSS | n = 32/47 | n = 39/47 | 0.50 |
| | 4.1^{†} (1.3-NR) | 1.1 (0.3-4.2) | (0.31, 0.80) |
| Non-HbSS | n = 11/20 | n = 15/18 | 0.47 |
| | 6.9 (1.4-NR) | 3.1 (1.1-6.2) | (0.21, 1.05) |

HU use

| | | | |
|---|---|---|---|
| Yes | n = 28/42 | n = 33/40 | 0.58 |
| | 2.4^{†} (1.2-NR) | 1.2 (0.3-4.9) | (0.35, 0.96) |
| No | n = 15/25 | n = 21/25 | 0.39 |
| | 5.7^{†} (3.1-NR) | 2.9 (0.8-4.5) | (0.20, 0.76) |

Data are median (interquartile range) unless otherwise stated.
n = number of patients in subpopulation who experienced an SCPC during the study/total number of patients in subpopulation.
*SelG1 5.0 mg/kg vs placebo;
^{†}Log-rank P value < 0.05.
CI, confidence interval; NR, not reportable.
HR, hazard ratio.

TABLE 7
Safety

| | SelG1 | | SelG1 | | | |
|---|---|---|---|---|---|---|
| | 5.0 mg/kg | | 2.5 mg/kg | | Placebo | |
| | (N = 66) | | (N = 64) | | (N = 62) | |
| Adverse Event† | no. (%) | | no. (%) | | no. (%) | |
| Patients with at least 1 | 17 | (25.8) | 21 | (32.8) | 17 | (27.4) |
| serious adverse event | | | | | | |
| Most frequent serious | | | | | | |
| adverse events‡ | | | | | | |
| Pyrexia | 2 | (3.0) | 0 | | 1 | (1.6) |
| Influenza | 0 | | 3 | (4.7) | 0 | |
| Pneumonia | 3 | (4.5) | 2 | (3.1) | 3 | (4.8) |
| Patients with at least | 57 | (86.4) | 56 | (87.5) | 55 | (88.7) |
| 1 adverse event | | | | | | |
| Most frequent | | | | | | |
| adverse events§ | | | | | | |
| Headache | 11 | (16.7) | 14 | (21.9) | 10 | (16.1) |
| Back pain | 10 | (15.2) | 13 | (20.3) | 7 | (11.3) |
| Nausea | 12 | (18.2) | 11 | (17.2) | 7 | (11.3) |
| Arthralgia | 12 | (18.2) | 9 | (14.1) | 5 | (8.1) |
| Pain in extremity | 11 | (16.7) | 8 | (12.5) | 10 | (16.1) |
| Urinary tract | 9 | (13.6) | 7 | (10.9) | 7 | (11.3) |
| infection | | | | | | |
| Upper respiratory | 7 | (10.6) | 7 | (10.9) | 6 | (9.7) |
| tract infection | | | | | | |
| Pyrexia | 7 | (10.6) | 6 | (9.4) | 4 | (6.5) |
| Diarrhoea | 7 | (10.6) | 5 | (7.8) | 2 | (3.2) |
| Musculoskeletal | 8 | (12.1) | 4 | (6.3) | 6 | (9.7) |
| pain | | | | | | |
| Pruritus | 5 | (7.6) | 7 | (10.9) | 3 | (4.8) |
| Vomiting | 5 | (7.6) | 7 | (10.9) | 3 | (4.8) |
| Chest pain | 1 | (1.5) | 7 | (10.9) | 1 | (1.6) |

*Includes randomized patients who received at least one dose of study drug;
†AEs were coded with the use of preferred terms from the MedDRA;
‡The event occurred in at least 2 patients in either active treatment group;
§The event occurred in at least 10% of patients in either active treatment group.

Sequences
P-selectin amino acid sequence
SEQ ID NO: 1
WTYHYSTKAYSWNISRKYCQNRYTDLVAIQNKNEIDYLNKVLPYY
SSYYWIGIRKNNKTWTWVGTKKALTNEAENWADNEPNNKRNNEDC
VEIYIKSPSAPGKWNDEHCLKKKHALCYTASCQDMSCSKQGECLE
TIGNYTCSCYPGFYGPECEYVRECGELELPQHVLMNCSHPLGNES
FNSQCSFHCTDGYQVNGPSKLECLASGIWTNKPPQCLAAQCPPLK
IPERGNMTCLHSAKAFQHQSSCSFSCEEGFALVGPEVVQCTASGV
WTAPAPVCK
CDR light chain amino acid sequence
SEQ ID NO: 2
KASQSVDYDGHSYMN
CDR light chain amino acid sequence
SEQ ID NO: 3
AASNLES
CDR light chain amino acid sequence
SEQ ID NO: 4
QQSDENPLT
Mature light chain variable region amino
acid sequence
SEQ ID NO: 5
DIQMTQSPSSLSASVGDRVTITCKASQSVDYDGHSYMNWYQQKPG
KAPKLLIYAASNLESGVPSRESGSGSGTDFTLTISSLQPEDFATY
YCQQSDENPLTFGGGTKVEIKR
CDR heavy chain amino acid sequence
SEQ ID NO: 6
SYDIN
CDR heavy chain amino acid sequence
SEQ ID NO: 7
WIYPGDGSIKYNEKEKG
CDR heavy chain amino acid sequence
SEQ ID NO: 8
RGEYGNYEGAMDY
Mature heavy chain variable region amino
acid sequence
SEQ ID NO: 9
QVQLVQSGAEVKKPGASVKVSCKVSGYTFTSYDINWVRQAPGKGL
EWMGWIYPGDGSIKYNEKFKGRVTMTVDKSTDTAYMELSSLRSED
TAVYYCARRGEYGNYEGAMDYWGQGTLVTVSS
Human Kappa constant region amino acid sequence
SEQ ID NO: 10
TVAAPSVFIFPPSDEQLKSGTASVVCLLNNFYPREAKVQWKVDNA
LQSGNSQESVTEQDSKDSTYSLSSTLTLSKADYEKHKVYACEVTH
QGLSSPVTKSENRGEC
IgG₂ constant region amino acid sequence
SEQ ID NO: 11
ASTKGPSVFPLAPCSRSTSESTAALGCLVKDYFPEPVTVSWNSGA
LTSGVHTFPAVLQSSGLYSLSSVVTVTSSNFGTQTYTCNVDHKPS
NTKVDKTVERKCCVECPPCPAPPVAGPSVFLFPPKPKDTLMISRT
PEVTCVVVDVSHEDPEVQFNWYVDGMEVHNAKTKPREEQFNSTFR
VVSVLTVVHQDWLNGKEYKCKVSNKGLPAPIEKTISKTKGQPREP
QVYTLPPSREEMTKNQVSLTCLVKGFYPSDIAVEWESNGQPENNY
KTTPPMLDSDGSFFLYSKLTVDKSRWQQGNVFSCSVMHEALHNHY
TQKSLSLSPGK
Light chain mature amino acid sequence
SEQ ID NO: 12
DIQMTQSPSSLSASVGDRVTITCKASQSVDYDGHSYMNWYQQKPG
KAPKLLIYAASNLESGVPSRFSGSGSGTDFTLTISSLQPEDFATY
YCQQSDENPLTFGGGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSENRGEC
Heavy chain mature amino acid sequence
SEQ ID NO: 13
QVQLVQSGAEVKKPGASVKVSCKVSGYTFTSYDINWVRQAPGKGL
EWMGWIYPGDGSIKYNEKFKGRVTMTVDKSTDTAYMELSSLRSED
TAVYYCARRGEYGNYEGAMDYWGQGTLVTVSSASTKGPSVFPLAP
CSRSTSESTAALGCLVKDYFPEPVTVSWNSGALTSGVHTFPAVLQ
SSGLYSLSSVVTVTSSNFGTQTYTCNVDHKPSNTKVDKTVERKCC
VECPPCPAPPVAGPSVFLFPPKPKDTLMISRTPEVTCVVVDVSHE
DPEVQENWYVDGMEVHNAKTKPREEQFNSTFRVVSVLTVVHQDWL
NGKEYKCAVSNKGLPAPIEKTISKTKGQPREPQVYTLPPSREEMT
KNQVSLTCLVKGFYPSDIAVEWESNGQPENNYKTTPPMLDSDGSF
FLYSKLTVDKSRWQQGNVFSCSVMHEALHNHYTQKSLSLSPGK
Light chain complete amino acid sequence
SEQ ID NO: 14
MESQTQVFVYMLLWLSGVDGDIQMTQSPSSLSASVGDRVTITCKA
SQSVDYDGHSYMNWYQQKPGKAPKLLIYAASNLESGVPSRESGSG
SGTDFTLTISSLQPEDFATYYCQQSDENPLTFGGGTKVEIKRTVA
APSVEIFPPSDEQLKSGTASVVCLLNNFYPREAKVQWKVDNALQS
GNSQESVTEQDSKDSTYSLSSTLTLSKADYEKHKVYACEVTHQGL
SSPVTKSENRGEC
Heavy chain complete amino acid sequence
SEQ ID NO: 15
MKCSWVIFFLMAVVTGVNSQVQLVQSGAEVKKPGASVKVSCKVSG
YTFTSYDINWVRQAPGKGLEWMGWIYPGDGSIKYNEKFKGRVTMT
VDKSTDTAYMELSSLRSEDTAVYYC
ARRGEYGNYEGAMDYWGQGTLVTVSSASTKGPSVFPLAPCSRSTS
ESTAALGCLVKDYFPEPVTVSWNSGALTSGVHTFPAVLQSSGLYS
LSSVVTVTSSNFGTQTYTCNVDHKPSNTKVDKTVERKCCVECPPC
PAPPVAGPSVELFPPKPKDTLMISRTPEVTCVVVDVSHEDPEVQF
NWYVDGMEVHNAKTKPREEQFNSTFRVVSVLTVVHQDWLNGKEYK
CAVSNKGLPAPIEKTISKTKGQPREPQVYTLPPSREEMTKNQVSL
TCLVKGFYPSDIAVEWESNGQPENNYKTTPPMLDSDGSFFLYSKL
TVDKSRWQQGNVFSCSVMHEALHNHYTQKSLSLSPGK
Complete light variable region amino
acid sequence
SEQ ID NO: 18
MESQTQVFVYMLLWLSGVDGDIQMTQSPSSLSASVGDRVTITCKA
SQSVDYDGHSYMNWYQQKPGKAPKLLIYAASNLESGVPSRFSGSG
SGTDFTLTISSLQPEDFATYYCQQSDENPLTFGGGTKVEIKR
Complete heavy variable region amino
acid sequence
SEQ ID NO: 19
MKCSWVIFFLMAVVTGVNSQVQLVQSGAEVKKPGASVKVSCKVSG
YTFTSYDINWVRQAPGKGLEWMGWIYPGDGSIKYNEKFKGRVTMT
VDKSTDTAYMELSSLRSEDTAVYYCARRGEYGNYEGAMDYWGQGT
LVTVSS
IgG₂ constant region amino acid sequence
with a one amino acid residue
mutation to reduce complement activation
SEQ ID NO: 23
ASTKGPSVFPLAPCSRSTSESTAALGCLVKDYFPEPVTVSWNSGA
LTSGVHTFPAVLQSSGLYSLSSVVTVTSSNFGTQTYTCNVDHKPS
NTKVDKTVERKCCVECPPCPAPPVAGPSVELFPPKPKDTLMISRT
PEVTCVVVDVSHEDPEVQFNWYVDGMEVHNAKTKPREEQFNSTFR
VVSVLTVVHQDWLNGKEYKCAVSNKGLPAPIEKTISKTKGQPREP
QVYTLPPSREEMTKNQVSLTCLVKGFYPSDIAVEWESNGQPENNY
KTTPPMLDSDGSFFLYSKLTVDKSRWQQGNVFSCSVMHEALHNHY
TQKSLSLSPGK

1.-18. (canceled)

19. A method of reducing the frequency of sickle cell pain crisis comprising administering crizanlizumab or a binding fragment thereof, wherein the antibody is first provided in a loading phase, during which a subject receives two loading doses of crizanlizumab or a binding fragment thereof at an amount of 2.5 mg/kg, 5 mg/kg, or 7.5 mg/kg and wherein the time interval between the two loading doses is 2 weeks (+/−3 days), and then further provided in a maintenance phase, during which the subject receives a plurality of maintenance doses of the antibody at an amount of 2.5 mg/kg, 5 mg/kg, or 7.5 mg/kg and wherein the time interval between the plurality of maintenance doses is 4 weeks (+/−3 days).

20. The method of claim 19, wherein crizanlizumab or a binding fragment thereof is first provided in a loading phase, during which the subject receives two loading doses of the antibody at an amount of 5 mg/kg and wherein the time interval between the two loading doses is 2 weeks (+/−3 days), and then further provided in a maintenance phase, during which the subject receives a plurality of maintenance doses of the antibody at an amount of 5 mg/kg and wherein the time interval between the plurality of maintenance doses is 4 weeks (+/−3 days).

21. The method of claim 19, wherein crizanlizumab or a binding fragment thereof is first provided in a loading phase, during which the subject receives two loading doses of the antibody at an amount of 7.5 mg/kg and wherein the time interval between the two loading doses is 2 weeks (+/−3 days), and then further provided in a maintenance phase, during which the subject receives a plurality of maintenance doses of the antibody at an amount of 7.5 mg/kg and wherein the time interval between the plurality of maintenance doses is 4 weeks (+/−3 days).

22. The method of claim 19, wherein crizanlizumab or a binding fragment thereof is administered in combination with hydroxyurea.

23. The method of claim 19, wherein crizanlizumab or a binding fragment thereof is provided to the subject by an intravenous route.


SEQUENCE LISTING (23 sequences)

SEQ ID NO: 1 - protein, 279 aa, Homo sapiens
WTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCK

SEQ ID NO: 2 - protein, 15 aa, synthetic construct
  REGION 1..15: note = CDR light chain amino acid sequence
KASQSVDYDGHSYMN

SEQ ID NO: 3 - protein, 7 aa, synthetic construct
  REGION 1..7: note = CDR light chain amino acid sequence
AASNLES

SEQ ID NO: 4 - protein, 9 aa, synthetic construct
  REGION 1..9: note = CDR light chain amino acid sequence
QQSDENPLT

SEQ ID NO: 5 - protein, 112 aa, synthetic construct
  REGION 1..112: note = Mature light chain variable region amino acid sequence
DIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKR

SEQ ID NO: 6 - protein, 5 aa, synthetic construct
  REGION 1..5: note = CDR heavy chain amino acid sequence
SYDIN

SEQ ID NO: 7 - protein, 17 aa, synthetic construct
  REGION 1..17: note = CDR heavy chain amino acid sequence
WIYPGDGSIKYNEKFKG

SEQ ID NO: 8 - protein, 13 aa, synthetic construct
  REGION 1..13: note = CDR heavy chain amino acid sequence
RGEYGNYEGAMDY

SEQ ID NO: 9 - protein, 122 aa, synthetic construct
  REGION 1..122: note = Mature heavy chain variable region amino acid sequence
QVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSS

SEQ ID NO: 10 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 326 aa, Homo sapiens
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 218 aa, synthetic construct
  REGION 1..218: note = Light chain mature amino acid sequence
DIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 448 aa, synthetic construct
  REGION 1..448: note = Heavy chain mature amino acid sequence
QVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 238 aa, synthetic construct
  REGION 1..238: note = Light chain complete amino acid sequence
MESQTQVFVYMLLWLSGVDGDIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 467 aa, synthetic construct
  REGION 1..467: note = Heavy chain complete amino acid sequence
MKCSWVIFFLMAVVTGVNSQVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16-17 - skipped (2 entries)

SEQ ID NO: 18 - protein, 132 aa, synthetic construct
  REGION 1..132: note = Complete light variable region amino acid sequence
MESQTQVFVYMLLWLSGVDGDIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKR

SEQ ID NO: 19 - protein, 141 aa, synthetic construct
  REGION 1..141: note = Complete heavy variable region amino acid sequence
MKCSWVIFFLMAVVTGVNSQVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSS

SEQ ID NO: 20-22 - skipped (3 entries)

SEQ ID NO: 23 - protein, 326 aa, synthetic construct
  REGION 1..326: note = IgG2 constant region amino acid sequence with a one amino acid residue mutation to reduce complement activation
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK